(19) United States
(12) Patent Application Publication
(10) Pub. No.: US 2002/0116734 A1
(43) Pub. Date: Aug. 22, 2002

(54) Plant derived bag homologues

(76) Inventor: Martin B. Dickman, Lincoln, NE (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 09/946,805

(22) Filed: Sep. 4, 2001

Related U.S. Application Data
(60) Provisional application No. 60/232,566, filed on Sep. 14, 2000.

(51) Int. Cl. A01H 5/00; C07H 21/04; C12N 9/00

(52) U.S. Cl. 800/278; 435/183; 435/69.1; 435/325; 435/320.1; 536/23.2; 435/410

(57) ABSTRACT

This invention provides isolated BAG polypeptides and functional fragments thereof, isolated nucleic acid molecules encoding the same, methods utilizing the polypeptides or functional fragments thereof, and transgenic plants or transgenic plant cells with altered expression level of BAG genes.

### CROSS-REFERENCE TO RELATED APPLICATIONS

This application claims the benefit of U.S. Provisional Patent Application No. 60/232,566, filed Sep. 14, 2000, which is incorporated herein by reference in its entirety.

### BACKGROUND OF THE INVENTION

1. Field of the Invention

This invention relates generally to nucleic acid molecules and products encoded thereby that modulate apoptosis in plants. More specifically, this invention discloses compositions comprising nucleic acids and polypeptides corresponding to BAG, an apoptotic pathway protein, methods of using the compositions to identify other apoptotic pathway proteins or to modulate apoptosis in a plant, and transgenic plants or plant cells with an altered level of BAG polypeptides.

2. Description of the Related Art

Programmed cell death (“apoptosis”) is the active process of cell death and has been found to be an intrinsic part of the development of animals (reviewed in Ellis et al., Ann. Rev. Cell Biol. 7:663-98, 1991; Raff, Nature 356:397-400, 1992; Vaux, Proc. Natl. Acad. Sci. USA 90:786-89, 1993; Steller, Science 267:1445-49, 1995). It plays an essential role in morphogenesis and in development of the immune system and the nervous system. Recently, apoptosis has also been shown to play an important role in tissue homeostasis, regulation of cell numbers, elimination of damaged or abnormal cells and defense against infections (Raff, Nature 356: 397-400, 1992; Ameisen, AIDS 8:1197-213, 1994; Vaux et al., Cell 76:777-79, 1994). Dysregulation of apoptosis, conversely, is involved in the pathogenesis of several human diseases, including cancer, autoimmunity, AIDS and neurodegenerative disorders (reviewed in Williams, Cell 65:1097-98, 1991; Nagata and Golstein, Science 267:1449-56, 1995; Thompson, Science 267:1456-62, 1995).

Apoptosis is mediated by a variety of diverse signals generated by apoptotic pathway gene products. The stimuli that regulate the functions of these apoptotic gene products include both extracellular and intracellular signals. Either the presence or the removal of a particular stimulus can be sufficient to evoke a positive or negative apoptotic signal. Physiological stimuli that inhibit or reduce the likelihood of apoptosis include, for example, growth factors, extracellular matrix, CD40 ligand, viral gene products, neutral amino acids, zinc, estrogen and androgens. In contrast, stimuli that promote apoptosis include, for example, tumor necrosis factor (TNF), Fas, transforming growth factor β (TGF-β), neurotransmitters, growth factor withdrawal, loss of extracellular matrix attachment, intracellular calcium and glucocorticoids. Other stimuli, including those of environmental and pathogenic origin, also exist and can either induce or inhibit apoptosis. The various stimuli result in complex interactions of cellular proteins and ultimately lead into a cell death pathway that is evolutionarily conserved between humans and invertebrates. The pathway includes a cascade of proteolytic zymogen activation events analogous to that of the blood coagulation cascade.

With recent advances in understanding the complex signaling pathways that induce programmed cell death in animal cells, research has intensified in identifying similar pathways in evolutionarily distant organisms, such as plants. In plants, programmed cell death-regimes are recognized to occur at specific points during development (such as during xylogenesis, reproduction, and senescence) (Drake et al., Plant Mol. Biol. 30:755-767, 1996; Greenberg, Proc. Natl. Acad. Sci. USA 93:12094-97, 1996) and may be triggered in response to pathogens (see, Woodson et al., Plant Physiol. 99:526-532, 1992; O'Neill et al., Plant Cell 5:419-432, 1993; Chasan, Plant Cell 6:917-919, 1994; Smart, New Phytol. 126:419-448, 1994; Keen, Ann. Rev. Genet. 24:447-463, 1990; Lamb, Cell 76:419-422, 1994; Mittler et al, Cell 7:29-42, 1995). Recent evidence suggests that plant cell death might be mechanistically similar to animal apoptosis in some cases such as in plant development, disease associated death, and hypersensitive reaction (Dickman et al., Proc. Natl. Acad. Sci. 98:6957-6962, 2001; Wang et al., Plant Cell 8:375-391, 1996; Ryerson and Heath, Plant Cell 8:393-402, 1996). The dying plant cells appear morphologically similar to apoptotic cells: They form apoptotic bodies and are accompanied by DNA cleavage often with the characteristics of endonucleolytically processed DNA (Wyllie, Curr. Opin. Gen. Dev. 5:97-104, 1995; Levin et al., Curr. Biol. 6:427-37, 1996; Wang et al., Plant Cell 8:375-391, 1996; Mittler et al. Plant Cell 7:29-42, 1995; Ryerson and Heath, Plant Cell 8:393-402, 1996). Despite these similarities between programmed cell death in plants and animals, some aspects of the function and mechanism of programmed cell death in plants may still differ from what is seen in animals. For example, plant cells do not engulf their dead neighbors. In some cases, the dead plant cells become part of the very architecture of the plant performing crucial functions such as xylem and phloem. Currently, very little is known about the genes and corresponding proteins that control programmed cell death in plants and which animal genes (vertebrate and invertebrate) that are involved in apoptosis have homologues in plants.

Accordingly, given the recognized importance of apoptosis in animals and its importance in development and pathogen resistance in plants, understanding plant apoptotic pathways is extremely valuable. Such understanding may lead to methods of regulating the pathway and generating transgenic plants harboring cell death modulators that have unique phenotypic characteristics, such as resistance to various biotic and abiotic insults, as well as increased shelf-life of cut plants, fruits, and vegetables.

The present invention describes BAGs, plant protein homologues of animal BAG proteins, identified as being involved in plant apoptosis. The present invention satisfies the needs for improving understanding of plant apoptosis pathway and provides methods of regulating plant cell death and generating transgenic plants with decreased level of senescence, biotic insult or abiotic insult resistance, and other beneficial features.

### BRIEF SUMMARY OF THE INVENTION

The present invention generally provides isolated BAG polypeptides and fragments thereof, isolated nucleic acid molecules encoding the same, assays utilizing BAG polypeptides or functional fragments thereof, as well as transgenic plants and plant cells with increased or decreased levels of BAG polypeptides. In one aspect, isolated nucleic acid molecules encoding BAG polypeptides are provided. In certain embodiments, the encoded polypeptides are plant BAG polypeptides. In related embodiments Arabidopsis thaliana BAG polypeptides are provided, such as A. thaliana BAG-1, BAG-2, BAG-3, and BAG-4. In yet other embodiments, the invention provides isolated nucleic acid sequences that hybridize with at least moderate stringency with SEQ ID NOS: 1, 3, 5, or 7, and nucleic acid molecules that encode polypeptides having at least 70% identity with SEQ ID NOS: 2, 4, 6, or 8. The complement of any one of the nucleic acid sequence described above is also included in the invention.

In another aspect, the invention provides nucleic acid molecules encoding a functional fragment of a BAG polypeptide having at least 70% identity with amino acids 131-210, amino acids 138-219, or amino acids 131-219 of the A. thaliana BAG-1 polypeptide (SEQ ID NO:2). In certain embodiments, the nucleotide sequence comprises nucleotides 496-735, nucleotides 517-762, or nucleotides 496-762 of SEQ ID NO: 1. In related embodiments, the nucleic acid molecule encodes amino acids 131-210, amino acids 138-219, or amino acids 131-219 of SEQ ID NO:2.

In a further aspect, the present invention provides an isolated nucleic acid molecule comprising a sequence that encodes a functional fragment having at least 70% identity with amino acids 48-109 or amino acids 48-118 of a BAG polypeptide of A. thaliana BAG-1 polypeptide (SEQ ID NO:2). In certain embodiments, the BAG domain is encoded by nucleotides 247-432 or nucleotides 247-459 of SEQ ID NO:1. In further embodiments, the nucleic acid molecule encodes amino acids 48-109 or amino acids 48-118 of SEQ ID NO:2.

In additional aspects, the present invention provides expression vectors encoding BAG polypeptides or functional fragments thereof. In certain embodiments, the BAG polypeptide and BAG functional fragments are encoded by the sequences recited above. These sequences are under the control of a constitutive promoter in some embodiments, while under the control of an inducible promoter or event specific promoter in other embodiments.

In a related aspect, host cells are provided which contain vectors encoding BAG or functional fragments thereof, as described above. The host cells may be essentially any cell, such as mammalian cells, yeast cells, plant cells, insect cells or bacterial cells.

The invention further provides isolated BAG polypeptides or functional fragments thereof wherein the polypeptide has at least 70% amino acid identity with SEQ ID NOS: 2, 4, 6, or 8. In certain embodiments, the BAG polypeptide or its functional fragment is capable of associating with a Bcl-2 related protein. In other embodiments, the polypeptide is a plant BAG. In yet other embodiments, the polypeptide is an A. thaliana BAG, such as A. thaliana BAG-1, BAG-2, BAG-3, or BAG-4 (SEQ ID NOS: 2, 4, 6, or 8, respectively). In additional embodiments, the functional fragment of an isolated BAG polypeptide comprises a polypeptide having at least 70% identity with amino acids 131-210, amino acids 138-219, or amino acids 131-219 of SEQ ID NO:2 or with amino acids 48-109 or amino acids 48-118 of SEQ ID NO:2.

Other aspects of the invention provide antibodies that specifically bind an isolated BAG polypeptide or functional fragment thereof. In some embodiments, the plant BAG polypeptide is an A. thaliana BAG polypeptide such as A. thaliana BAG-1, BAG-2, BAG-3, and BAG-4 (SEQ ID NOS: 2, 4, 6, and 8, respectively). In other embodiments, the functional fragment of a BAG polypeptide comprises amino acids 131-210, amino acids 138-219, amino acids 131-219, amino acids 48-109, or amino acids 48-118 of SEQ ID NO:2. In yet other embodiments, the antibody modulates apoptosis. In additional embodiments, the antibody is a polyclonal, a monoclonal, a chimeric antibody, a single chain antibody, or a humanized antibody, or an antigen-binding fragment.

In yet additional aspects, the present invention provides methods of detecting apoptotic pathway proteins. In one embodiment the method comprises contacting a sample with a BAG polypeptide or a functional fragment thereof under conditions that permit formation of complex between the BAG polypeptide or functional fragment thereof and the apoptotic pathway protein, and detecting the complex and the apoptotic pathway protein in the complex. In certain embodiments, a detectable moiety is covalently bound to the BAG polypeptide or its functional fragments for the detection of the complex. The detectable moiety may be a reporter molecule (such as Glutathione-S-transferase (GST), green fluorescent protein (GFP), or the like) or a radionuclide. The apoptotic pathway proteins detected may include a caspase, a rev-caspase, Bcl-2, Bcl-2 family members, Apaf-1, Bad, Bax, Ced-9, Ced-4, and HSP70. In some embodiments, the sample comprises a cDNA expression library.

In another aspect, the invention provides methods for modulating apoptosis in a plant comprising transforming cells of the plant with a nucleic acid molecule comprising a promoter functional in cells of the plant and operably linked to a nucleic acid sequence encoding a BAG polypeptide or its functional fragment, regenerating the plant cells to provide a differentiated plant, and identifying a transformed plant which expresses the coding sequence and exhibits altered apoptosis. The promoter may be an inducible plant promoter such as those inducible by a plant pathogen, a constitutive plant promoter, a tissue-specific promoter, or an event specific promoter.

In a related aspect, a transgenic plant or transgenic plant cell is provided that contains and expresses a transgene comprising a nucleic acid encoding a BAG polypeptide or its functional fragment operably linked to a promoter. In another related aspect, a transgenic plant or transgenic plant cell is provided that contains and expresses a transgene comprising a nucleic acid encoding a BAG polypeptide or its functional fragment linked to a promoter in the antisense orientation. In certain embodiments, both types of transgenic plants or transgenic plant cells may be selected from the group consisting of graminaceae, solanaceae, rosaceae, compositeae, leguminaceae, brassicaceae, and cucurbitaceae. The promoter may be a plant inducible promoter such as those inducible by a plant pathogen, a constitutive promoter, or a tissue-specific promoter. In certain embodiments, these transgenic plants are biotic insult resistant where the biotic insult may be induced by an insect, by a plant pathogen such as a fungus, a nematode, a bacterium, or a virus, or by another living organism. In other embodiments, the transgenic plants are abiotic insult resistant where the abiotic insult may be induced by high moisture, low moisture, salinity, nutrient deficiency, air pollution, high temperature, low temperature, soil toxicity, herbicides, insecticides, or other stress conditions. In yet other embodiments, at least a portion of the transgenic plants exhibits a decreased level of senescence. In some embodiments, transgenic plant cells may be protoplasts, gamete producing cells and cells capable of regenerating into a whole plant.

In another aspect, the present invention provides methods for screening for a compound that modulates (i.e., increases or decreases) the specific binding between a BAG polypeptide and a BAG binding protein, comprising (a) combining the BAG polypeptide with the BAG binding protein in the absence of a candidate compound under conditions that allow specific binding between the BAG polypeptide and the BAG binding protein; (b) combining the BAG polypeptide with the BAG binding protein in the presence of the candidate compound under the conditions of step (a); and (c) comparing the specific binding between the BAG polypeptide and the BAG binding protein of step (a) with that of step (b) to thereby determine whether the candidate compound is capable of modulating the specific binding between the BAG polypeptide and the BAG binding protein. In certain embodiments, the BAG polypeptide is an isolated BAG polypeptide comprising a polypeptide that has at least 70% amino acid identity with Arabidopsis BAG1 (SEQ ID NO:2), BAG2 (SEQ ID NO:4), BAG3 (SEQ ID NO:6) or BAG4 (SEQ ID NO:8), or a functional fragment of the isolated BAG polypeptide. Preferably, either the BAG polypeptide or the BAG binding protein is covalently bound to a detectable moiety, including but not limited to a reporter molecule and radionuclide.

In a related aspect, the present invention provides methods for screening for a compound that disrupts the binding between a BAG polypeptide and a BAG binding protein comprising (a) contacting a candidate compound with a binding complex comprising the BAG polypeptide and the BAG binding protein; and (b) detecting either the BAG polypeptide or the BAG binding protein that dissociates from the binding complex to thereby determine whether the candidate compound is capable of disrupts the binding between the BAG polypeptide and the BAG binding protein. In certain embodiments, the BAG polypeptide is an isolated BAG polypeptide comprising a polypeptide that has at least 70% amino acid identity with Arabidopsis BAG1 (SEQ ID NO:2), BAG2 (SEQ ID NO:4), BAG3 (SEQ ID NO:6) or BAG4 (SEQ ID NO:8), or a functional fragment of the isolated BAG polypeptide. Preferably, either the BAG polypeptide or the BAG binding protein is covalently bound to a detectable moiety, including but not limited to a reporter molecule and radionuclide.

These and other aspects of the present invention will become evident upon reference to the following detailed description and attached drawings. In addition, the various references set forth herein describe in more detail certain procedures or compositions (e.g. plasmids, etc.), and are therefore incorporated by reference in their entirety.

### BRIEF DESCRIPTION OF THE SEVERAL VIEWS OF THE DRAWINGS

FIG. 1 represents the nucleotide sequence of A. thaliana BAG-1 of SEQ ID NO:1.

FIG. 2 represents the amino acid sequence of A. thaliana BAG-1 of SEQ ID NO:2.

FIGS. 3A and 3B represent the nucleotide sequence of A. thaliana BAG-2 of SEQ ID NO:3.

FIG. 4 represents the amino acid sequence of A. thaliana BAG-2 of SEQ ID NO:4.

FIG. 5 represents the nucleotide sequence of A. thaliana BAG-3 of SEQ ID. NO:5.

FIG. 6 represents the amino acid sequence of A. thaliana BAG-3 of SEQ ID NO:6.

FIGS. 7A and 7B represent the nucleotide sequence of A. thaliana BAG-4 of SEQ ID NO:7.

FIG. 8 represents the amino acid sequence of A. thaliana BAG-4 of SEQ ID NO:8.

FIG. 9 depicts a binding assay in which radiolabeled A. thaliana BAG-1 protein was incubated with a GST-HSP70 fusion protein or a GST-Bcl-2 fusion protein bound to Sepharose beads. Lane 1 shows GST-HSP70 incubated with ³⁵S-BAG-1. Lane 2 shows GST-Bcl-2 incubated with ³⁵S-BAG-1. Lane 3 shows ³⁵S-BAG-1 incubated with Sepharose beads alone as a control. Lane 4 shows the in vitro translation of BAG-1.

FIG. 10 is a schematic representation of exemplary predicted functional domains of A. thaliana BAG-1 of SEQ ID NO:1.

FIGS. 11A and 11B are scanned images representing Northern blot analysis of A. thaliana BAG-1 gene (SEQ ID NO:1) and HSP 70 gene in various tissues of A. thaliana plants, respectively.

FIGS. 12A and 12B are scanned images representing electrophoresis analysis (FIG. 12A) of PCR products from wild type Arabidopsis genomic DNA (WT A.t.), tobacco genomic DNA (Glurk), and genomic DNA of transgenic tobacco plants containing an Arabidopsis BAG gene and its corresponding autoradiograph (FIG. 12B) after probed with an Arabidopsis BAG clone.

### DETAILED DESCRIPTION OF THE INVENTION

The following definitions of certain terms used in the description of the invention are provided to be helpful in understanding the invention.

An “apoptotic pathway protein”, as used herein, refers to any one of the proteins that regulate, disrupt the regulation of, or otherwise are involved directly or indirectly in the programmed cell death pathway as has been elucidated in a number of organisms, including human, mouse, C. elegans, Drosophila Melanogaster, and includes baculovirus proteins. In addition, the phrases “apoptotic pathway protein encoding gene” or “apoptotic pathway gene” are used interchangeably herein. A variety of apoptotic pathway proteins are known and are useful within the context of the present invention. Exemplary molecules include caspase molecules (currently 14 of which have been identified: caspase-1 to caspase-14), bcl-2 family members, Bax, Bak, Bok, Bad, Bik, Bid, Blk, Hrk, BNIP3, BimL, EGL-1, Inhibitors of Apoptosis (e.g., IAP-baculovirus, DIAP 1 & 2-Drosophila, c-IAP 1 & 2-human, X-IAP-human, NIAP-human, survivin-human, etc.), and the like. The sequences for these and other genes are available from the Genbank Database and are described in numerous publications including When Cells Die, edited by Lockshin et al., Wiley-Liss, New York, 1998.

The term “Bcl-2 related proteins” or “Bcl-2 family members” refers to proteins that are structurally and functionally related to Bcl-2. Such proteins have at least weak sequence homology with Bcl-2 and are able to regulate apoptosis and share similar domains. They include, but are not limited to, Bcl-2, Bcl-B, Bcl-X_{L}, Bcl-X_{S}, Bcl-W, McL-1, A1, NR-13, Ced-9, E1B 19K, BHRF1, KSHV ORF 16, LMW5-HL, KS-Bcl-2, etc.

A “BAG polypeptide” is a polypeptide that is able to associate in vitro or in vivo with an apoptotic pathway protein, and has at least 40% sequence similarity with a human BAG (Takayama et al., Genomics 35:494-498, 1996) in a region of the human BAG at least 75 amino acids in length. As used herein, the term “associate” refers to the ability of a BAG polypeptide and an apoptosis-related protein to have a binding affinity for each other such that the BAG and the apoptosis-related protein form a bound complex. The affinity of binding of a BAG polypeptide and an apoptosis-related protein is sufficiently specific such that the bound complex can form in vivo in a cell or can form in vitro under appropriate conditions, as described herein. The formation or dissociation of a bound complex can be identified as described in Example 3 or using other well-known methods such as equilibrium dialysis. Examples of apoptotic pathway proteins known to associate with a BAG polypeptide include Bcl-2 related proteins, Raf-1, receptors for hepatocyte growth factor (HGF), platelet-derived growth factor (PDGF), glucocorticoid, estrogen, thyroid hormone and retinoic acid, and heat shock protein HSP70.

Within the context of this invention, a “BAG polypeptide” includes wild-type protein sequences, as well as other variants (including alleles) of the native protein sequence. Such variants may result from natural polymorphisms or may be synthesized by recombinant methodology, and differ from wild-type protein by one or more amino acid substitutions, insertions, deletions, or the like. Typically, when engineered, amino acid substitutions will be conservative, i.e., substitution of amino acids within groups of polar, non-polar, aromatic, or charged, amino acids. In the region of homology to the native sequence, variants should have at least 70% identity, and preferably 80%, and more preferably 85%, most preferably at least 90% amino acid sequence identity, and within certain embodiments, greater than 92%, 95%, or 97% identity, and all integer values in between 70-100%. Such amino acid sequence identity may be determined by standard methodologies, including use of the National Center for Biotechnology Information BLAST search methodology available at www.ncbi.nlm.nih.gov using default parameters. The identity methodologies most preferred are those described in U.S. Pat. No. 5,691,179 and Altschul et al., Nucleic Acids Res. 25:3389-3402, 1997, or any FASTA or Smith-Waterman based algorithm using default parameters, incorporated herein by reference.

A “functional fragment,” an “isolated domain,” or a “domain” of a BAG polypeptide is a portion of a BAG polypeptide that retains the ability to associate with an apoptotic pathway protein. Exemplary functional fragments include, but are not limited to amino acids 131-210, amino acids 138-219, amino acids 131-219, amino acids 48-109, or amino acids 48-118 of Arabidopsis BAG-I (SEQ ID NO:2).

An “isolated nucleic acid molecule” refers to a polynucleotide molecule in the form of a separate fragment or as a component of a larger nucleic acid construct, that has been separated from its source cell (including the chromosome it normally resides in) at least once, and preferably in a substantially pure form. Nucleic acid molecules may be comprised of a wide variety of nucleotides, including DNA, RNA, nucleotide analogues, or a combination thereof.

A “complement” of a particular nucleotide sequence is a contiguous nucleotide sequence that forms Watson-Crick base pairs with that particular nucleotide sequence.

A “nucleic acid vector”, as used herein, refers to a DNA molecule such as a plasmid, cosmid, or bacteriophage that has the capability of replicating autonomously in a host cell, including both a cloning vector and an expression vector. A cloning vector typically contains one or a small number of restriction endonuclease recognition sites at which foreign DNA sequences can be inserted in a determinable fashion without loss of essential biological function of the vector, as well as a marker gene suitable for use in the identification and selection of cells transformed with the vector. An expression vector is a DNA molecule having a gene that is expressed in a host cell. Typically gene expression is placed under the control of certain regulatory elements including promotes, tissue-specific regulatory elements and enhancers.

A “promoter”, as used herein, refers to a DNA sequence that directs the transcription of a gene. Typically a promoter is located in the 5′ region of a gene, proximal to the transcriptional start site of a structural gene. A promoter is functional in plant cells if it is able to direct expression of a gene in plant cells. A promoter is constitutive if it directs transcription of a gene under most environmental conditions and states of development or cell differentiation. A promoter is inducible if it is capable of directly or indirectly activating transcription of a nucleic acid sequence in response to an inducer. A tissue-specific promoter is a promoter that directs transcription of a gene in a specific plant tissue or tissues. An event specific promoter is a promoter that is active or up-regulated only upon the occurrence of an event, such as tumorigenicity or viral infection.

The term “operably linked” refers to functional linkage between a promoter sequence and a structural gene regulated by the promoter sequence. A “structural gene” refers to a DNA sequence that is transcribed into messenger RNA (mRNA) which is then translated an amino acid sequence. The operably linked promoter controls the expression of the structural gene. The term “expression” generally refers to the transcription of a DNA sequence.

A “host cell” refers to a cell that contains a nucleic acid vector.

The term “modulate” refers to the ability to alter from basal level. As used in the context of apoptosis (e.g., “modulate apoptosis”), “modulate” refers to the ability to alter or change any biochemical, physiological or morphological events associated with apoptosis from its basal level. For example, apoptosis has been modulated if the expression of a gene involved in apoptotic pathway, the interaction of an apoptotic pathway protein with other proteins, the formation of apoptotic bodies, or the DNA cleavage is altered from its original state.

The term “sample”, as used herein, refers to any compositions suspected of containing an apoptotic pathway protein, including but not limited to cell extracts, cDNA expression libraries and recombinant proteins.

A “caspase” refers to a cysteine protease with specificity for substrate cleavage at Asp-X bonds, where “X” is an amino acid.

As used herein, a “rev-caspase” refers to an engineered cysteine protease that specifically cleaves proteins after Asp residues and is expressed as a zymogen, in which a small subunit is N-terminal to a large subunit (see, e.g., PCT WO 99/00632).

A “yeast two-hybrid screening system” is a system for identifying polypeptide sequences that bind to a predetermined polypeptide sequence through reconstitution of a transcriptional factor in yeast cells. A “transcriptional factor”, as used herein, refers to a protein containing a DNA-binding domain that binds to a regulatory element of a gene and a transcription activation domain that activates the transcription of the gene. Nucleic acids encoding two hybrid proteins, one consisting of a DNA-binding domain of a transcription factor (e.g., Gal 4) fused to the nucleic acid sequence of a known protein and the other consisting of a transcription activation domain of the transcription factor fused to the nucleic acid of a second protein, are constructed and introduced into a yeast host cell. Alternatively, the transcription activation domain of the transcription factor may be fused to the known protein and the DNA-binding domain of the transcription factor may be fused to the second protein. Intermolecular binding between the two fusion proteins reconstitutes the DNA-binding domain with the transcription activation domain, which leads to the transcriptional activation of a reporter gene (e.g., lacZ, His3) operably linked to the DNA binding domain of the transcription factor.

A “biotic insult”, as used herein, refers to plant challenge caused by viable or biologic agents (biotic agents). Examples of biotic agents that cause a biotic insult include, insects, fungi, bacteria, viruses, nematodes, viroids, mycloplasmas, etc.

An “abiotic insult”, as used herein, refers to plant challenge by a non-viable or non-living agent (abiotic agent). Examples of abiotic agents that cause an abiotic insult include environmental factors such as low moisture (drought), high moisture (flooding), nutrient deficiency, radiation levels, air pollution (ozone, acid rain, sulfur dioxide, etc.), high temperature (hot extremes), low temperature (cold extremes), and soil toxicity, as well as herbicide damage, pesticide damage, or other agricultural practices (e.g., over-fertilization, improper use of chemical sprays, etc.).

As used herein, a “plant pathogen”, refers to any agent that causes a disease state in a plant, including but not limited to viruses, fungi, bacteria, nematodes, and other related microorganisms.

As used herein, the term “plant” refers to a whole plant, including a plantlet. Suitable plants for use in the invention include any plants amenable to transformation techniques, including both monocotyledonous and dicotyledonous plants.

Examples of monocotyledonous plants include, but are not limited to, asparagus, field and sweet corn, barley, wheat, rice, sorghum, onion, pearl millet, rye and oat, and ornamentals. Examples of dicotyledonous plants include, but are not limited to, tomato, potato, arabidopsis, tobacco, cotton, rapeseed, field beans, soybeans, peppers, lettuce, peas, alfalfa, clover, cole crops or Brassica oleracea (e.g., cabbage, broccoli, cauliflower, Brussels sprouts), radish, carrot, beets, eggplant, spinach, cucumber, squash, melons, cantaloupe, sunflowers and various ornamentals.

The term “plant cell”, as used herein, refers to protoplasts or cells derived from a plant, including gamete producing cells and cells which are capable of regenerating into whole plants. Accordingly, a seed comprising multiple plant cells and cultured cells derived from various plant tissues that are capable of regenerating into a whole plant is included in the definition of “plant cell”.

As used herein, “plant tissue” includes differentiated and undifferentiated tissues of a plant, including but not limited to roots, stems, shoots, leaves, pollen, seeds, tumor tissue and various forms of cells and culture such as single cells, protoplasts, embryos, and callus tissue.

The term “transgene” or “heterologous nucleic acid sequence”, as used herein, refers to a nucleic acid sequence containing at least one structural gene generally linked with a regulatory sequence such as a promoter. A heterologous nucleic acid sequence is a sequence that originates in a foreign species, or in the same species if substantially modified from its original form. The term “transgene” includes a nucleic acid originating in the same species, where such sequence is operably associated with a promoter that differs from the natural or wild-type promoter.

A gene is linked to a promoter “in the antisense orientation” if it is linked to the promoter in such a way that the transcript of the gene is complementary to the mRNA capable of being translated into the polypeptide product of the gene. Therefore, no polypeptide product of the gene is made when the gene is linked to the promoter in the antisense orientation.

BAG Nucleic Acid Molecules

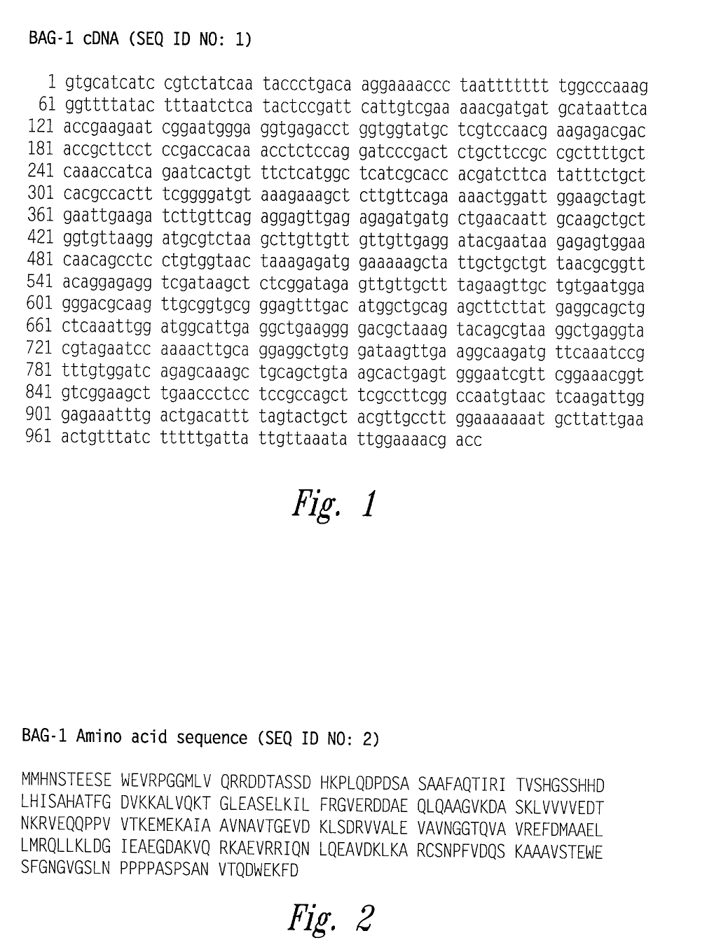

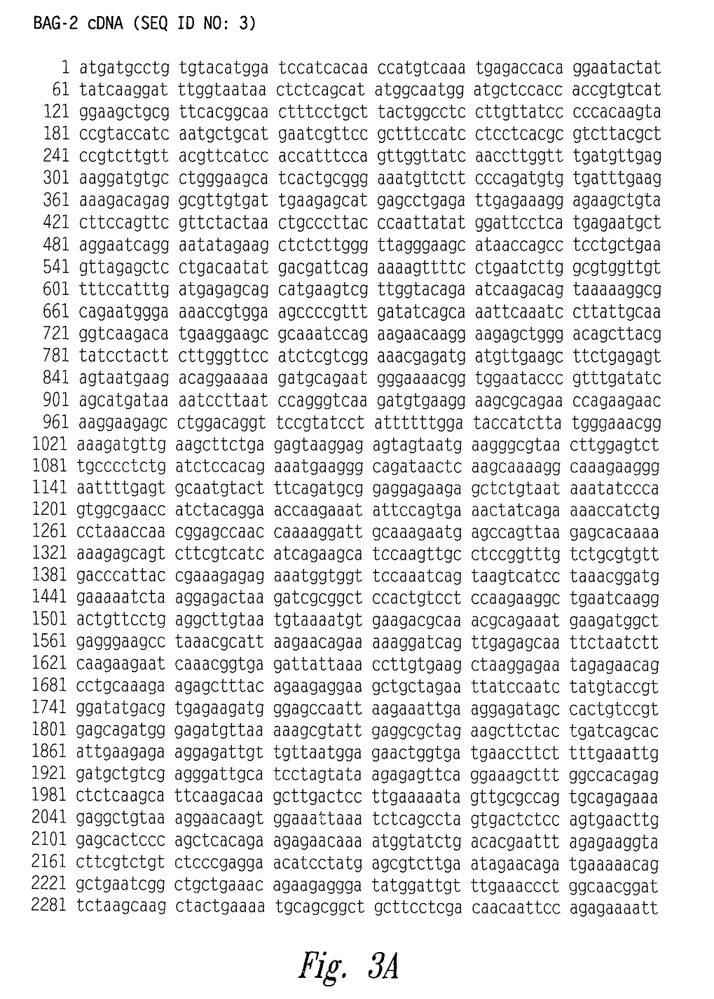

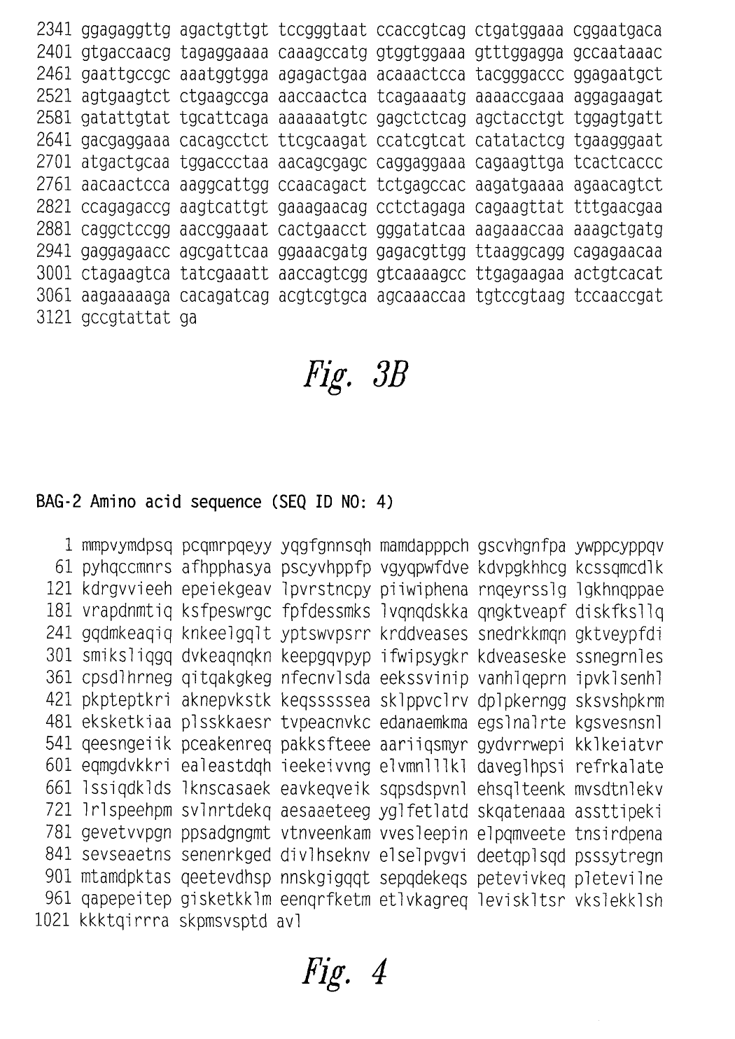

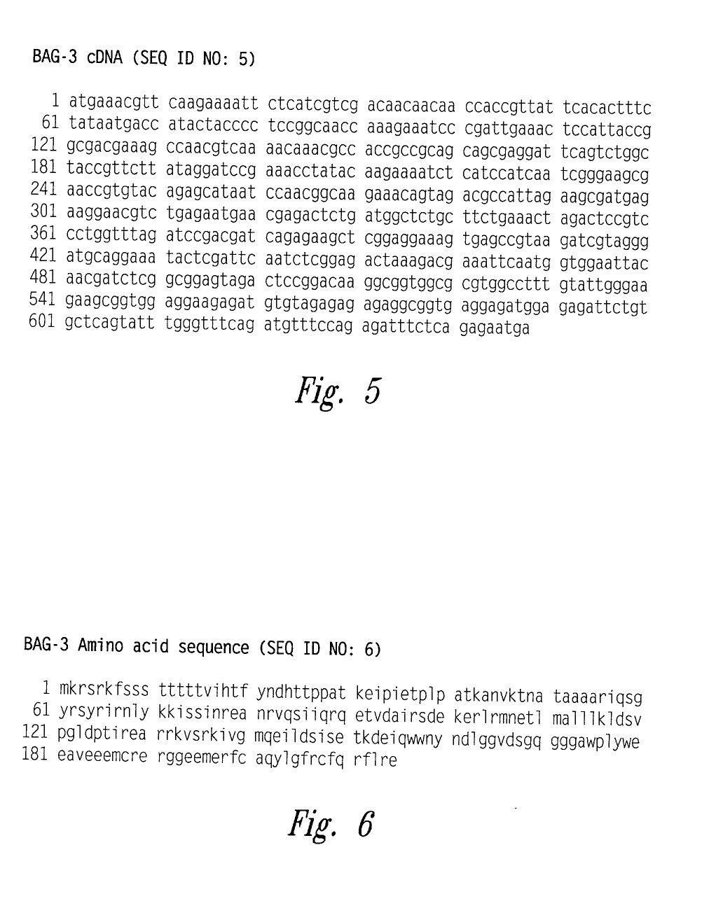

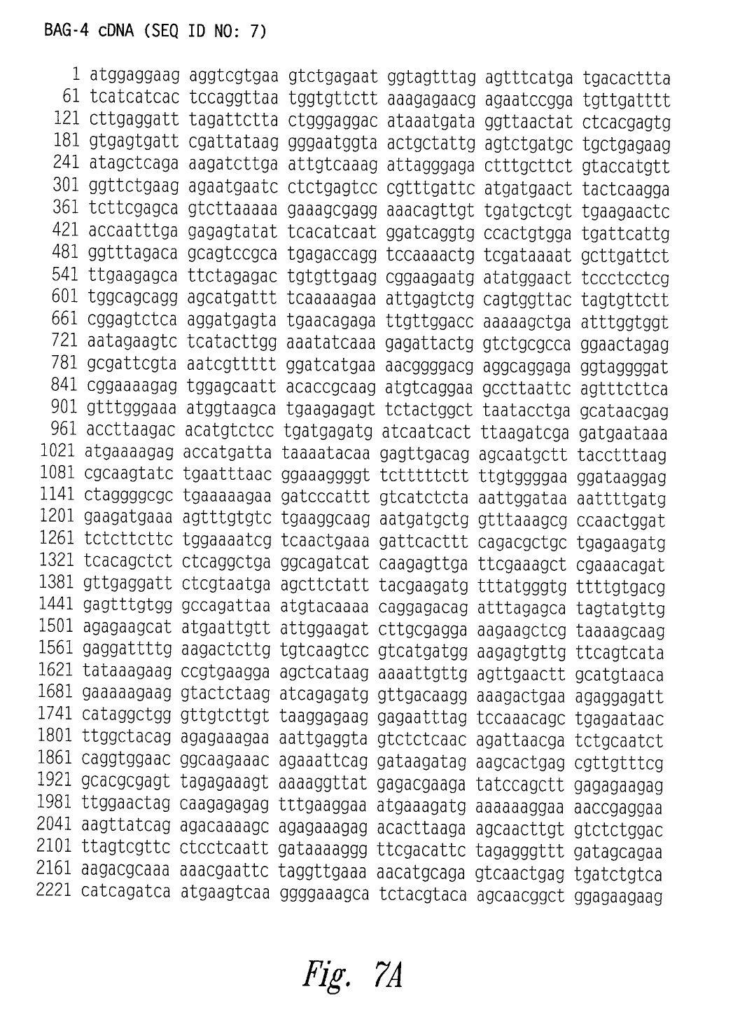

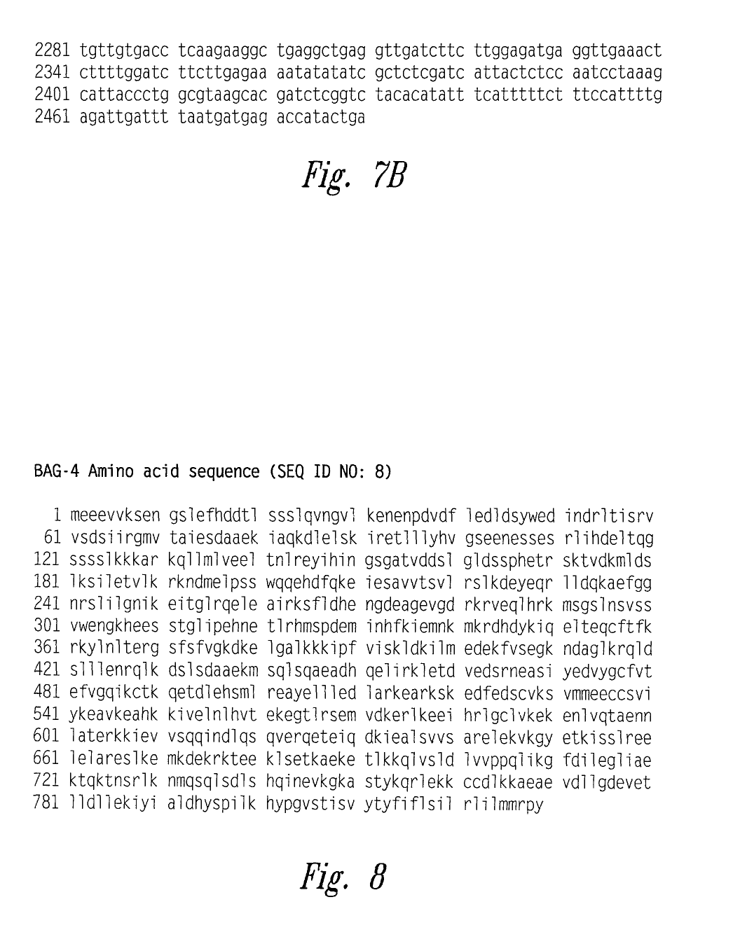

The present invention provides nucleic acid molecules that encode a homologue of animal BAG. The invention discloses nucleic acid sequences that encode a plant BAG polypeptide, such as A. thaliana BAG-1, BAG-2, BAG-3, and BAG-4, or functional fragments thereof. The four Arabidopsis BAG genes were isolated using a profile-profile alignment method (Rychlewski, L., et al., 2000 Protein Sci. 2:232-41). Briefly, this method was used to search Genbank database and four BAG homologues in A. thaliana (Genbank database, gi3068705, gi3702325, gi3157923 and gi3132472) were identified. Two primers, 5′-gggaggtgagacctggtgg-3′ (SEQ ID NO:9) and 5′-ggccgaaggcgaagctggcgg-3′ (SEQ ID NO:10), were made according to one of the sequence (i.e., gi3068705) and used to generate a PCR fragment from genomic A. thaliana DNA that corresponded to gi3068705. The resulting PCR fragment was used to probe a cDNA library (cDNA library CD4-7, Arabidopsis Biological Resource Center (see Newman, et al., Plant Physiol. 106:1241-55; 1994)) for its corresponding full-length cDNA. The above technique was also used for obtaining the full-length cDNA clones of the other three Arabidopsis homologues. The nucleic acid sequences of the other homologues (i.e., BAG-2, -3, -4) are shown in FIGS. 3, 5 and 7, respectively, whereas the amino acid sequences of these homologues are shown in FIGS. 4, 6, and 8, respectively. The predicted amino acid sequence of A. thaliana BAG-1 (FIG. 2) contains conserved domains known in mammalian BAGs (FIG. 10).

Besides the four Arabidopsis BAG genes, the invention also includes all nucleic acid sequences that encode BAG polypeptides, or functional fragments thereof that are substantially homologous with SEQ ID NOS: 2, 4, 6, or 8. A BAG polypeptide is substantially homologous with SEQ ID NOS: 2, 4, 6, or 8 if the degree of homology between the BAG polypeptide with SEQ ID NO:2, 4, 6 or 8 is greater than that between the BAG polypeptide and any other previously reported BAG polypeptides (e.g., human and mouse BAGs) and is derived from plant tissue. In certain embodiments, a BAG polypeptide that is substantially homologous with SEQ ID NOS: 2, 4, 6 or 8 is at least 50%, 60%, 70%, 75%, 80%, 90%, or 95% similar to any one of SEQ ID NOS: 2, 4, 6 and 8.

Nucleic acid molecules of the invention are less than a whole chromosome and can be single- or double-stranded. Preferably, the polynucleotide molecules are intron-free. Nucleic acid molecules of the invention can comprise at least 11, 15, 18, 21, 30, 33, 42, 54, 60, 66, 72, 84, 90, 100, 120, 140, 160, 180, 200, 220, 240, 250, 260, 300, 330, 400, 420, 500, 540, 600, 660, 700, or more contiguous nucleotides selected from A. thaliana BAG genes (SEQ ID NOS: 1, 3, 5, and 7), the homologues of these genes, and the complements of the genes and the homologues as well as degenerate forms.

The BAG nucleic acid molecules of the current invention include all native nucleic acids encoding polypeptides or functional fragments thereof having substantial similarity with an A. thaliana BAG polypeptide (e.g., SEQ ID NOS: 2, 4, 6, or 8), including natural variants of BAG genes (e.g., degenerate forms, polymorphisms, splice variants or mutants). These molecules may be identified by a variety of methods. For instance, higher order algorithms such as those in profile-profile alignment methods, Pileup and Blast programs can be used to probe gene databases for nucleic acid sequences having weak homology with known BAG genes. The identified sequences may subsequently be used to design PCR primers for the amplification of such sequences. For instance, two barley BAG homologues (i.e., GenBank Accession Nos. 7613222 and 7611205) were identified using tblastn search of translated EST database (est_others) with an Arabidopsis BAG-1 clone (i.e., GenBank Accession No. 3068705).

Nucleic acids encoding polypeptides having substantial sequence identity with A. thaliana BAGs (SEQ ID NOS:2, 4, 6, and 8) may be isolated from either the genomic DNA or preferably the cDNA of the organisms. Construction of both genomic and cDNA libraries are well known (see, e.g., Sambrook et al., Molecular Cloning: A Laboratory Manual, Cold Spring Harbor Press, 1989). Briefly, cDNA libraries can be constructed in bacteriophage vectors (e.g., XZAPII), plasmids, or others suitable for screening, while genomic DNA libraries can be constructed in chromosomal vectors, such as YACs (yeast artificial chromosomes), bacteriophage vectors (e.g., λEMBL3, Xgt10), cosmids, or plasmids. In addition, certain genomic and cDNA libraries are commercially available (e.g., cDNA library CD4-7 from the Arabidopsis Biological Resource Center).

Nucleic acids encoding A. thaliana BAGs (SEQ ID NOS: 1, 3, 5, and 7) or fragments thereof may be used as probes for screening genomic or cDNA libraries for sequences encoding A. thaliana BAG homologues. To facilitate hybridization detection, the sequences or fragments may be labeled with a reporter molecule, such as a radionuclide, (e.g. ³²P), enzymatic label, protein label, fluorescent label, or biotin. The genomic or cDNA libraries are generally plated as phage plaques or bacterial colonies, depending upon the vector used, and transferred to nitrocellulose or nylon membranes. These membranes are then hybridized with the labeled probes. Clones that hybridize with the probes may be verified as containing BAG DNA by any of known means including, for example, DNA sequence analysis of the inserts of the positive clones or hybridization with a second, non-overlapping probe. Isolated inserts of the positive clones may contain full-length cDNA and genomic DNA encoding BAG polypeptides. Alternatively, full-length sequences may be obtained by either re-screening the same libraries or other libraries from the same organisms as the original libraries are.

Nucleic acids that encode polypeptides or functional fragments thereof having substantial sequence identity with A. thaliana BAGs may also be obtained by PCR amplification with portions of A. thaliana BAG genes as primers. The amplified sequences may be verified to contain sequences encoding BAG homologues by DNA sequencing analysis. Full-length genomic or cDNA sequences encoding the homologues may be obtained by screening corresponding genomic or cDNA libraries.

The above nucleic acids may also be made using the techniques of synthetic chemistry given the sequences disclosed herein or identified with methods described above. In addition, the degeneracy of the genetic code permits alternate nucleotide sequences which will encode the amino acid sequences presented in SEQ ID NOS: 2, 4, 6, and 8. All such nucleotide sequences are within the scope of the present invention.

This invention also includes variant nucleic acids or functional fragments thereof that encode polypeptides having substantial sequence identity with A. thaliana BAGs, but are not present naturally in any organism. These non-natural variant nucleic acids may encode natural polypeptides with a deletion, insertion or substitution and may be made by either synthetic chemistry or any known mutagenesis techniques. Many methods for generating mutants have been developed (see generally, Ausubel et al., Current Protocols in Molecular Biology, Greene Publishing, 1995). Preferred methods include alanine scanning mutagenesis and PCR generation of mutants using an oligonucleotide containing the desired mutation to amplify mutant nucleic acid molecules. The variant nucleic acids of this invention generally have at least 70% or 75% nucleotide identity to the native sequence, preferably at least 80%-85%, and most preferably at least 90%, 95%, or 98% nucleotide identity with SEQ ID NOS:1, 3, 5, or 7. The identity algorithms and settings that may be used are defined herein supra, including computer programs which employ the Smith-Waterman algorithm, such as the MPSRCH program (Oxford Molecular), using an affine gap search with the following parameters: a gap open penalty of 12 and a gap extension penalty of 1. Preferably, GCG PileUp program (Genetics Computer Group, Madison, Wis.) (Gapweight: 4, Gaplength weight: 1) is used for sequence alignment. Furthermore, typically, a variant nucleic acid is sufficiently similar in sequence (preferably contains 15-25% basepair mismatches, even more preferably 5-15% basepair mismatches) to be able to hybridize to the reference sequence under moderate or stringent hybridization conditions.

Typically, for stringent hybridization conditions, a combination of temperature and salt concentration should be chosen that is approximately 12-20° C. below the calculated Tm of the hybrid under study. The Tm of a hybrid between a nucleotide sequence as shown in SEQ ID NOS: 1, 3, 5, or 7 and a polynucleotide sequence which is 70%, 75%, 80%, 85%, 90%, 95%, 96%, 97%, 98%, or 99% identical to SEQ ID NOS: 1, 3, 5, or 7 can be calculated, for example, using the equation of Bolton and McCarthy, Proc. Natl. Acad. Sci. U.S.A. 48, 1390 (1962):

Tₘ=81.5° C.−16.6(log₁₀[Na⁺])+0.41(%G+C)−0.63(%formamide)−600/l),

where l=the length of the hybrid in basepairs.

Examples of moderately stringent conditions include prewashing in a solution of 5× SSC, 0.5% SDS, 1.0 mM EDTA (pH 8.0); hybridizing at 50° C.-65° C., in 5× SSC, overnight; followed by washing twice at 65° C. for 20 minutes with each of 2×, 0.5× and 0.2× SSC containing 0.1% SDS. Stringent wash conditions include, for example, 4× SSC at 65° C., or 50% formamide, 4× SSC at 42° C., or 0.5× SSC, 0.1% SDS at 65° C. Highly stringent wash conditions include, for example, 0.2× SSC at 65° C. The wash conditions used to identify homologous sequences containing at most about 25-30% basepair mismatches may be as follows: 2× SSC (0.3 M NaCl, 0.03 M sodium citrate, pH 7.0), 0.1% SDS, room temperature twice, 30 minutes each; then 2× SSC, 0.1% SDS, 50° C. once, 30 minutes; then 2× SSC, room temperature twice, 10 minutes each.

This invention also includes nucleic acids encoding natural BAG polypeptides or functional fragments thereof, but having substituted codons optimized for expression in a given host cell. In addition, the invention includes nucleic acids that encode polypeptides that are recognized by antibodies that bind a BAG polypeptide or fragment thereof. These nucleic acids may be made using synthetic chemistry or mutagenesis techniques described above.

Nucleic acid sequences encoding BAG polypeptides or functional fragments thereof may be fused to sequences encoding a secretion signal, whereby the resulting polypeptide is a precursor protein that is subsequently processed and secreted. The resulting processed BAG polypeptide may be recovered from the cell lysate, periplasmic space, phloem, or from the growth or fermentation medium. Secretion signals suitable for use are widely available and well known in the art (e.g., von Heijne, J. Mol. Biol. 184:99-105, 1985).

Nucleic acids encoding BAG polypeptides or functional fragments thereof may also be fused to any other sequences known to be useful for facilitating the purification or detection of the encoded BAG polypeptides. These sequences include, but are not limited to, histidine (His) tag, Flag fragment, GFP, maltose-binding protein (MBP), and GST.

This invention also includes nucleic acid molecules that contain nucleic acid encoding BAG polypeptides or functional fragments thereof fused to either a DNA-binding domain or a transcription activation domain of a transcription factor. Vectors containing such molecules may be used to identify proteins that interact with BAG polypeptides, such as apoptotic pathway proteins, as described infra.

In certain embodiments, compositions and methods of the invention include ribozymes, antisense RNA and dominant-negative BAG mutants to decrease levels of functional BAG polypeptides. Ribozymes are trans-cleaving catalytic RNA molecules possessing endoribonuclease activity. Ribozymes may be specifically designed for a particular target nucleotide sequence. Ribozymes may be engineered to cleave an RNA species site-specifically in the background of cellular RNA. The cleavage event renders the mRNA unstable and prevents protein expression. Preparation and usage of ribozymes is well known to the art (see Usman et al, Current Opin. Struct. Biol. 6:527-533, 1996; Long et al., FASEB J 7:25, 1993; Symons, Ann. Rev. Biochem. 61:641, 1992 and U.S. Pat. No. 5,254,678). Accordingly, the BAG nucleic acid sequences provided by the invention allows for the construction of an effective BAG ribozyme.

The present invention also provides antisense nucleic acids for BAG genes such as the complements of the nucleotide sequences shown in SEQ ID NOS:1, 3, 5, and 7 or variants thereof. Usually, antisense nucleic acids are designed to specifically bind to RNA, resulting in the formation of RNA-DNA or RNA-RNA hybrids and an arrest in DNA replication, reverse transcription or messenger RNA translation. Antisense polynucleotides based on a selected sequence can specifically interfere with expression of the corresponding gene. They are typically generated within the cell by expression from antisense constructs that contain the antisense strand as the transcribed strand. Antisense production and uses thereof are discussed extensively in the literature and are widely known and available to one skilled in the art. (see Agrwal et al., Tet. Lett. 28:3539-3542, 1987; Miller et al., J. Am. Chem. Soc. 93:6657-6665, 1971; Stec et al., Tet. Lett. 26:2191-2194, 1985; Moody et al., Nucl. Acids Res. 12:4769-4782, 1989; Eckstein, Trends Biol. Sci. 14:97-100, 1989; Stein In: Oligodeoxynucleotides: Antisense Inhibitors of Gene Expression, Cohen, Ed, Macmillan Press, London, pp. 97-117, 1989; U.S. Pat. Nos. 5,168,053; 5,190,931; 5,135,917; 5,087,617; and 5,176,996). Effective BAG antisense expression vectors may be produced based on the BAG nucleic acid sequences provided by the invention, but typically will include at least 10 contiguous nucleotides. In certain embodiments no more than 100 contiguous nucleotides of BAG are used, while in other embodiments between 18-60 or between 20-50 contiguous nucleotides of BAG are utilized.

Nucleic acid molecules of the invention further include molecules that encode single-chain antibodies that specifically bind to the disclosed proteins.

Bag Polypetides and Functional Fragments Thereof

The present invention provides BAG polypeptides or functional fragments thereof that are identical or substantially homologous with SEQ ID NOS: 2, 4, 6, 8 and fragments thereof. Such BAG polypeptides include native BAG polypeptides that are naturally present in organisms, non-native BAG polypeptides, or BAG polypeptide fusion proteins. Native BAG polypeptides include all the variants resulting from polymorphism, alternative mRNA splicing/transcription, or differential polypeptide processing. Non-native BAG polypeptides may contain an amino acid deletion, insertion, or substitution of native BAG polypeptides. BAG polypeptide fusion proteins can be BAG polypeptides or functional fragments thereof fused with any other known proteins or portions thereof.

As defined above, a BAG polypeptide is substantially homologous with SEQ ID NOS: 2, 4, 6, or 8 if the degree of homology between the BAG polypeptide with SEQ ID NO:2, 4, 6 or 8 is greater than that between the BAG polypeptide and any other previously reported BAG polypeptides (e.g., human and mouse BAGs). Typically, in the region of homology to SEQ ID NOS: 2, 4, 6, or 8, BAG polypeptides or functional fragments thereof would have at least 70% amino acid identity, and preferably 80%, and more preferably 85%, most preferably at least 90% amino acid sequence identity, and within certain embodiments, greater than 92%, 95%, or 97% identity, and all integer values in between 70-100%. Such amino acid sequence identity may be determined by standard methodologies, as described supra. The functional fragments of BAG polypeptides of the invention typically are large enough to function as a binding domain for an antibody or for another apoptotic pathway protein and usually have at least 10, preferably 25, more preferably 40, in certain embodiments, 50 or 60 amino acids that are substantially homologous to SEQ ID NOS: 2, 4, 6, or 8.

For non-native BAG polypeptides or functional fragments thereof, guidance in determining which amino acid residues can be substituted, inserted, or deleted without abolishing biological or immunological activity can be found using computer programs well known in the art, such as DNASTAR software. Preferably, amino acid changes are conservative. A conservative amino acid change involves substitution of one amino acid for another amino acid of a family of amino acids with structurally related side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified as aromatic amino acids. Non-naturally occurring amino acids can also be used to form protein variants of the invention.

The present invention also encompasses the use of mutant and variant forms of genes or polypeptides, including dominant negative mutants. Dominant negative mutations are readily generated for a variety of proteins, including those that are active in homo- or heteromeric complexes. A mutant polypeptide will interact with wild-type binding partners and form a non-functional multimer or a multimer with altered, decreased or enhanced function. Thus, a preferred mutation is in a binding domain, a catalytic domain, or a cellular localization domain. Preferably, the dominant-negative polypeptide will be overproduced compared to wild type expression. Point mutations and deletions may be constructed which have such an effect. In addition, fusion of different polypeptides of various lengths to the terminus of a protein can yield dominant negative mutants (see Herskowitz, Nature 329:219-222, 1987).

BAG fusion proteins of the invention include polypeptides comprising BAG polypeptides or fragments thereof fused to amino acid sequences comprising one or more heterologous polypeptides. Such heterologous polypeptides may correspond to naturally occurring polypeptides of any source or may be recombinantly engineered amino acid sequences. Fusion proteins are useful for purification, generating antibodies against amino acid sequences, and for use in various assay systems. For example, fusion proteins can be used to identify proteins which interact with a protein of the invention or which interfere with its biological function. Fusion proteins comprising a signal sequence and/or a transmembrane domain of one or more of the disclosed proteins can be used to target other protein domains to cellular locations in which the domains are not normally found, such as bound to a cellular membrane or secreted extracellularly.

The polypepetides that BAG polypeptides or functional fragments thereof fused to can be full-length proteins or polypeptide fragments. Proteins commonly used in fusion protein construction include β-galactosidase, β-glucuronidase, GFP, blue fluorescent protein (BFP), GST, luciferase, horseradish peroxidase (HRP), and chloramphenicol acetyltransferase (CAT). Additionally, epitope tags can be used in fusion protein constructions, including His tags, FLAG tags, influenza hemagglutinin (HA) tags, Myc tags, VSV-G tags, and thioredoxin (Trx) tags. Other fusion constructions can include MBP, S-tag, Lex A DNA binding domain (DBD) fusions, GAL4 DNA binding domain fusions, and herpes simplex virus (HSV) BP16 protein fusions.

An isolated BAG polypeptide or functional fragment thereof can be obtained by a variety of methods known in the art. For example, a BAG polypeptide can be isolated by biochemical methods such as affinity chromatography. Affinity matrices that can be used for BAG isolation can be a solid phase having attached thereto anti-BAG monoclonal or polyclonal antibodies prepared against a BAG polypeptide or a functional fragment thereof comprising a BAG epitope. Alternatively, polypeptides known to bind BAG (e.g., Bcl-2) can be used as affinity matrices to isolate a BAG polypeptide or functional fragment thereof.

Other biochemical methods for isolating a BAG polypeptide or functional fragment thereof include preparative gel electrophoresis, gel filtration, affinity chromatography, ion exchange and reversed phase chromatography, chromatofocusing, isoelectric focusing and sucrose or glycerol density gradients (Deutscher, Methods in Enzymology: Guide to Protein Purification, Vol. 182, Academic Press, Inc., San Diego, Chapter 38, 1990; Balch et al., Methods in Enzymology, Vol 257, Academic Press, Inc., San Diego, Chapter 8, 1995). For example, a BAG polypeptide or functional fragment thereof can be isolated by preparative polyacrylamide gel electrophoresis and elution by diffusion or electroelution (Deutscher, supra, Chapter 33, 1990). Continuous elution gel electrophoresis using a system such as the Model 491 Prep Cell (BioRad, Hercules, Calif.) can be used to isolate a BAG polypeptide or functional fragment thereof. If desired, continuous elution gel electrophoresis can be combined with further purification steps such as liquid phase preparative isoelectric focusing using, for example, the Rotofor system (BioRad).

A BAG polypeptide or functional fragment thereof also can be produced by chemical synthesis, for example, by the solid phase peptide synthesis method (Merrifield et al., J. Am. Chem. Soc. 85:2149, 1964). Standard solution methods well known in the art also can be used to synthesize a BAG polypeptide or functional fragment thereof (Bodanszky, Principles of Peptide Synthesis, Springer-Verlag, Berlin, 1984; Bodanszky, Peptide Chemistry, Springer-Verlag, Berlin, 1993). A newly synthesized BAG polypeptide or functional fragment thereof can be isolated, for example, by high performance liquid chromatography and can be characterized using mass spectrometry or amino acid sequence analysis.

In addition, analogs of BAG or BAG peptides can be designed to have increased stability in vivo or in vitro or higher or lower affinity of binding to a Bcl-2-related protein by incorporating, for example, (D)-amino acids into a BAG peptide or by chemically modifying reactive amino acid side chains or the amino or carboxy terminus of a peptide. For example, a reactive amino group in a peptide can be rendered less reactive by acetylation. Furthermore, a modification such as acetylation changes a hydrophilic group to a hydrophobic group, which can be advantageous, when it is desirable to prepare a BAG peptide that can readily traverse a cell membrane.

A BAG polypeptide or functional fragment thereof can also be produced by recombinant DNA methods. Nucleic acids encoding BAG polypeptides or functional fragments thereof provided by the invention can be cloned into an appropriate vector for expression. Such a vector is commercially available or can be constructed by those skilled in the art and contains expression elements necessary for the transcription, translation, regulation, and, if desired, sorting of the BAG polypeptide or functional fragment thereof. The selected vector can also be used in a procaryotic or eukaryotic host system, as appropriate, provided the expression and regulatory elements are of compatible origin. A recombinant BAG polypeptide or functional fragment thereof produced in a host cell or secreted from the cell can be isolated using, for example, affinity chromatography with an anti-BAG antibody, ionic exchange chromotography, HPLC, size exclusion chromatography, ammonium sulfate crystallization, electrofocusing, or preparative gel electrophoresis (see generally Ausubel et al., supra; Sambrook et al., supra). An isolated purified protein is generally evidenced as a single band on an SDS-PAGE gel stained with Coomassie Blue.

BAG fusion polypeptides of the invention can be made by covalently linking two protein segments or by standard procedures in the art of molecular biology. For example, recombinant DNA methods can be used to prepare fusion proteins by making a DNA construct which comprises coding sequences selected from SEQ ID NOS:1, 3, 5, or 7 in proper reading frame with nucleotides encoding the second protein segment and expressing the DNA construct in a host cell, as is known in the art. Many kits for constructing fusion proteins are available from companies that supply research labs with tools for experiments, including, for example, Promega Corporation (Madison, Wis.), Stratagene (La Jolla, Calif.), Clontech (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

As defined above, the BAG polypeptides or functional fragments thereof have the ability to associate in vitro or in vivo an apoptotic pathway protein. Such apoptotic pathway proteins include, but are not limited to Bcl-2 related proteins, Raf-1, HGF receptors, PDGF receptors, receptors for glucocorticoid, estrogen, thyroid hormone and retinoic acid, and HSP70. The association of BAG polypeptides or functional fragments thereof with apoptotic pathway proteins may be detected or verified by any known protein-protein interaction assays. Examples of such assays include yeast two-hybrid systems, co-immunoprecipitation, in vitro binding assays on solid support, interaction cloning, and far western blot analysis.

BAG Binding Molecules

Also contemplated by the present invention are peptides, polypeptides, and other non-peptide molecules that specifically bind to a BAG polypeptide or a functional fragment thereof. As used herein, a molecule is said to “specifically bind” to BAG if it reacts at a detectable level with BAG, but does not react detectably with peptides containing an unrelated sequence. In certain embodiments, the dissociation constant of the interaction between a BAG binding molecule and a BAG polypeptide or a functional fragment thereof is at most 10-7 M. In other embodiments, the dissociation constant is at most 10-8 M. Preferred binding molecules include antibodies, which may be, for example, polyclonal, monoclonal, single chain, chimeric, or CDR-grafted antibodies, or fragments thereof, such as proteolytically generated or recombinantly produced F(ab′)₂, Fab, Fv, and Fd fragments. Certain preferred antibodies are those antibodies that inhibit or block BAG activity within an in vitro assay, as described herein. Binding properties of the antibody to BAG may generally be assessed using immunodetection methods including, for example, an enzyme-linked immunosorbent assay (ELISA), immunoprecipitation, and immunoblotting, which may be readily performed by those having ordinary skill in the art.

Methods well known in the art may be used to generate antibodies, polyclonal antisera, or monoclonal antibodies that are specific for BAG. Antibodies also may be produced as genetically engineered immunoglobulins (Ig) or Ig fragments designed to have desirable properties. For example, by way of illustration and not limitation, antibodies may include a recombinant IgG that is a chimeric fusion protein having at least one variable (V) region domain from a first mammalian species and at least one constant region domain from a second distinct mammalian species. Most commonly, a chimeric antibody has murine variable region sequences and human constant region sequences. Such a murine/human chimeric immunoglobulin may be “humanized” by grafting the complementarity determining regions (CDRs), which confer binding specificity for an antigen, derived from a murine antibody into human-derived V region framework regions and human-derived constant regions. Fragments of these molecules may be generated by proteolytic digestion, or optionally, by proteolytic digestion followed by mild reduction of disulfide bonds and alkylation, or by recombinant genetic engineering techniques.

Antibodies may generally be prepared by any of a variety of techniques known to those having ordinary skill in the art. See, e.g., Harlow et al., Antibodies: A Laboratory Manual, Cold Spring Harbor Laboratory, 1988. In one such technique, an animal is immunized with a BAG polypeptide or functional fragment thereof as antigen to generate polyclonal antisera. Suitable animals include rabbits, sheep, goats, pigs, cattle, and may include smaller mammalian species, such as mice, rats, and hamsters.

An immunogen may be comprised of cells expressing a BAG polypeptide or portions thereof, purified or partially purified BAG polypeptides or functional fragments thereof. BAG functional fragments may be generated by proteolytic cleavage or may be chemically synthesized. The present disclosure provides examples of nucleic acid sequences encoding BAG polypeptides and functional fragments thereof, such that those skilled in the art may routinely prepare them for use as immunogens. Peptides for immunization may also be selected by analyzing the primary, secondary, and tertiary structure of BAG according to methods known to those skilled in the art in order to determine amino acid sequences more likely to generate an antigenic response in a host animal. See, e.g., Novotny, Mol. Immunol. 28:201-207, 1991; Berzoksky, Science 229:932-40, 1985.

Monoclonal antibodies that specifically bind to BAG polypeptides or functional fragments thereof may be prepared, for example, using the technique of Kohler and Milstein (Nature, 256:495-497, 1975; Eur. J Immunol. 6:511-519, 1976) and improvements thereto. Hybridomas, which are immortal eukaryotic cell lines, may be generated that produce antibodies having the desired specificity to a BAG polypeptide or a fragment thereof. An animal—for example, a rat, hamster, or preferably mouse—is immunized with BAG immunogen prepared as described above. Lymphoid cells, most commonly, spleen cells, obtained from an immunized animal may be immortalized by fusion with a drug-sensitized myeloma cell fusion partner, preferably one that is syngeneic with the immunized animal. The spleen cells and myeloma cells may be combined for a few minutes with a membrane fusion-promoting agent, such as polyethylene glycol or a nonionic detergent, and then plated at low density on a selective medium that supports the growth of hybridoma cells, but not myeloma cells. A preferred selection media is HAT (hypoxanthine, aminopterin, thymidine). After a sufficient time, usually about 1 to 2 weeks, colonies of cells are observed. Single colonies are isolated, and antibodies produced by the cells may be tested for binding activity to the BAG polypeptide or variant or fragment thereof. Hybridomas producing antibody with high affinity and specificity for the BAG antigen are preferred. Hybridomas that produce monoclonal antibodies that specifically bind to a BAG polypeptide or variant or fragment thereof are contemplated by the present invention.

Within certain embodiments, the use of antigen-binding fragments of antibodies may be preferred. Such fragments include Fab fragments or F(ab′)₂ fragments, which may be prepared by proteolytic digestion with papain or pepsin, respectively. The antigen binding fragments may be separated from the Fe fragments by affinity chromatography, for example, using immobilized protein A or immobilized BAG polypeptide or a functional fragment thereof. An alternative method to generate Fab fragments includes mild reduction of F(ab′)₂ fragments followed by alkylation. See, e.g., Weir, Handbook of Experimental Immunology, 1986, Blackwell Scientific, Boston, 1986.

An additional method for selecting antibodies that specifically bind to a BAG polypeptide or a functional fragment thereof is by phage display. See, e.g., Winter et al., Annul. Rev. Immunol. 12:433-55, 1994; Burton et al., Adv. Immunol. 57:191-280, 1994. Human or murine immunoglobulin variable region gene combinatorial libraries may be created in phage vectors that can be screened to select Ig fragments (Fab, Fv, sFv, or multimers thereof) that bind specifically to a BAG polypeptide or a functional fragment thereof. See, e.g., U.S. Pat. No. 5,223,409; Huse et al., Science 246:1275-81, 1989; Kang et al., Proc. Natl. Acad. Sci. USA 88:4363-66, 1991; Hoogenboom et al., J. Molec. Biol. 227:381-388, 1992; Schlebusch et al., Hybridoma 16:47-52, 1997 and references cited therein. For example, polynucleotide sequences encoding Ig variable region fragments may be inserted into the genome of a filamentous bacteriophage, such as M13 or a variant thereof, in frame with the sequence encoding a phage coat protein, for example, gene III or gene VIII of M13, to create a fusion protein. A fusion protein may be a fusion of the coat protein with either the light chain variable region domain or the heavy chain variable region domain. Upon the identification of the variable region domain that specifically binds to a BAG polypeptide or a functional fragment thereof, nucleic acid molecules that encode antibodies or antigen-binding fragments of antibodies may be generated. Expression of such molecules in a host cell may decrease the level of functional BAG polypeptides, thus moderate apoptosis in the cell.

Vectors, Host Cells and Means of Expressing and Producing Protein

The present invention encompasses vectors comprising regulatory elements linked to BAG nucleic acid sequences. Such vectors may be used, for example, in the propagation and maintenance of BAG nucleic acid molecules, or in the expression and production of BAG polypeptides and nucleic acid molecules. Vectors may include, but are not limited to, plasmids, episomes, baculovirus, retrovirus, lentivirus, adenovirus, and parvovirus including adeno-associated virus.

Nucleic acid molecules encoding BAG polypeptides or functional fragments thereof may be expressed in a variety of host organisms. In certain embodiments, BAG are produced in mammalian cells, such as CHO, COS-7, or 293 cells. Other suitable host organisms include bacterial species (e.g., E. coli and Bacillus), other eukaryotes such as yeast (e.g., Saccharomyces cerevisiae), plant cells and insect cells (e.g., Sf9). Vectors for these hosts are well known in the art.

A DNA sequence encoding a BAG polypeptide or a functional fragment thereof is introduced into an expression vector appropriate for the host. The sequence is derived from an existing clone or synthesized. A preferred means of synthesis is amplification of the gene from cDNA, genomic DNA, or a recombinant clone using a set of primers that flank the coding region or the desired portion of the protein. Restriction sites are typically incorporated into the primer sequences and are chosen with regard to the cloning site of the vector. If necessary, translational initiation and termination codons can be engineered into the primer sequences. The BAG nucleic acid sequence can be codon-optimized for expression in a particular host. For example, a BAG gene isolated from an A. thaliana cell that is expressed in a fungal host, such as yeast, can be altered in nucleotide sequence to use codons preferred in yeast. Further, it may be beneficial to insert a traditional AUG initiation codon at the CUG initiation positions to maximize expression, or to place an optimized translation initiation site upstream of the CUG initiation codon. Accordingly, such codon-optimization may be accomplished by methods such as splice overlap extension, site-directed mutagenesis, automated synthesis, and the like.

At minimum, the vector must contain a promoter sequence. As used herein, a “promoter” refers to a nucleotide sequence that contains elements that direct the transcription of a linked gene. At minimum, a promoter contains an RNA polymerase binding site. More typically, in eukaryotes, promoter sequences contain binding sites for other transcriptional factors that control the rate and timing of gene expression. Such sites include TATA box, CAAT box, POU box, API binding site, and the like. Promoter regions may also contain enhancer elements. The promoter may be in the form of a promoter which is naturally associated with the gene of interest. Alternatively, the nucleic acid may be under control of a heterologous promoter not normally associated with the gene. In certain instances, the promoter elements may drive constitutive or inducible expression of the nucleic acid of interest.

The expression vectors used herein include a promoter designed for expression of the proteins in a host cell. Such promoters for expression in bacteria include promoters from the T7 phage and other phages, such as T3, T5, and SP6, and the trp, lpp, and lac operons. Hybrid promoters (see U.S. Pat. No. 4,551,433), such as tac and trc, may also be used. Promoters for expression in eukaryotic cells include the P10 or polyhedron gene promoter of baculovirus/insect cell expression systems (see, e.g., U.S. Pat. Nos. 5,243,041, 5,242,687, 5,266,317, 4,745,051, and 5,169,784), MMTV LTR, CMV IE promoter, RSV LTR, SV40, metallothionein promoter (see, e.g., U.S. Pat. No. 4,870,009), 35S promoter of CaMV, alcohol dehydrogenase gene promoter, chitinase gene promoter, and the like.

The promoter controlling transcription of a BAG nucleic acid may itself be controlled by a repressor. In some systems, the promoter can be derepressed by altering the physiological conditions of the cell, for example, by the addition of a molecule that competitively binds the repressor, or by altering the temperature of the growth media. Preferred repressor proteins include, but are not limited to, the E. coli lacI repressor responsive to IPTG induction, the temperature sensitive λcI857 repressor, and the like. The E. coli lacI repressor is preferred.

In other preferred embodiments, the vector also includes a transcription terminator sequence. A “transcription terminator region” has either a sequence that provides a signal that terminates transcription by the polymerase that recognizes the selected promoter and/or a signal sequence for polyadenylation.

Preferably, the vector is capable of replication in the host cells. Thus, when the host cell is a bacterium, the vector preferably contains a bacterial origin of replication. Preferred bacterial origins of replication include the f1-ori and col E1 origins of replication, especially the ori derived from pUC plasmids. In yeast, ARS or CEN sequences can be used to assure replication. A well-used system in mammalian cells is SV40 ori.

The plasmids also preferably include at least one selectable marker that is functional in the host. A selectable marker gene includes any gene that confers a phenotype on the host that allows transformed cells to be identified and selectively grown. Suitable selectable marker genes for bacterial hosts include the ampicillin resistance gene (Ampr), tetracycline resistance gene (Tcr) and the kanamycin resistance gene (Kan^{r}). The kanamycin resistance gene is presently preferred. Suitable markers for eukaryotes usually require a complementary deficiency in the host (e.g., thymidine kinase (tk) in tk-hosts). However, drug markers are also available (e.g., G418 resistance and hygromycin resistance).

The nucleic acid sequences encoding BAG polypeptides or functional fragments thereof, may also include a secretion signal, whereby the resulting peptide is a precursor protein that is processed and secreted. The resulting processed protein may be recovered from the periplasmic space, the growth medium, phloem, etc. Prokaryotic and eukaryotic secretion signals that are functional in E. coli, yeast, animal or plant cell may be employed. Such secretion signals are widely available and are well known in the art (See, e.g., Lei et al., J. Bacterial. 169:4379, 1987; von Heijne, J. Mol. Biol. 184:99-105, 1985; During et al., Plant Mol. Biol. 15:281-293, 1990; Lindsey and Jones in: Plant Cell Line Selection, pp. 317-335, VCH Weinham, Germany, 1990; Gruber and Crosby in: Methods in Plant Molecular Biology and Biotechnology, pp. 89-117, CRC Press, Boca Raton, Fla., 1993).

One skilled in the art appreciates that there are a wide variety of suitable vectors for expression in bacterial cells that are readily obtainable. Vectors such as the pET series (Novagen, Madison, Wis.), the tac and trc series (Pharmacia, Uppsala, Sweden), pTTQ18 (Amersham International plc, England), pACYC 177, the pGEX series, and the like are suitable for expression of BAG-1. Baculovirus vectors, such as pBlueBac (see, e.g., U.S. Pat. Nos. 5,278,050, 5,244,805, 5,243,041, 5,242,687, 5,266,317, 4,745,051, and 5,169,784; available from Invitrogen, San Diego) may be used for expression in insect cells, such as Spodoptera frugiperda sf9 cells (see U.S. Pat. No. 4,745,051). The choice of a bacterial host for the expression of BAG nucleic acid sequences is dictated in part by the vector. Commercially available vectors are paired with suitable hosts.

A wide variety of suitable vectors for expression in eukaryotic cells are also available. Such vectors include pCMVLacI and pXT1 available from Stratagene Cloning Systems (La Jolla, Calif.), and pCDNA series, pREP series, and pEBVHis available from Invitrogen (Carlsbad, Calif.). In certain embodiments, a BAG nucleic acid molecule is cloned into a gene targeting vector, such as pMC1 neo and a pOG series vector (Stratagene Cloning Systems).

The invention also contemplates that nucleic acid molecules encoding BAG polypeptides or its fragments may be inserted in-frame into plant vectors. General descriptions of plant expression vectors and reporter genes can be found in Gruber et al., “Vectors for Plant Transformation, in Methods in Plant Molecular Biology & Biotechnology” in Glich et al., Eds. pp. 89-119, CRC Press, 1993. Moreover, GUS expression vectors and GUS gene cassettes are available from Clontech Laboratories, Inc. (Palo Alto, Calif.), while GFP expression vectors and GFP gene cassettes are available from Aurora Biosciences (San Diego, Calif.).

The introduction of a vector into various cells, such as bacterial, yeast, insect, mammalian, and plant cells, are well known. For example, a vector can be transformed into a bacterial cell by heat shock or electroporation. Transformation of a yeast cell with a vector may also be carried out by eletroporation. Methods for introduction of vectors into animal cells include calcium phosphate precipitation, electrosporation, dextran-mediated transfection, liposome encapsulation, nucleus microinjection, and viral or phage infection. The introduction of heterologous nucleic acid sequences into plant cells can be achieved by particle bombardment, electroporation, microinjection, and Agrobacterium-mediated gene insertion (for reviews of such techniques, see, for example, Weissbach & Weissbach, Methods for Plant Molecular Biology, Academic Press, NY, Section VHI, pp. 421-463; 1988; Grierson & Corey, Plant Molecular Biology, 2d Ed., Blackie, London, Ch. 7-9, 1988, and Horsch et al., Science 227:1229, 1985; and Gene Transfer to Plants, eds. Potrykus. Springer Verlaa, 1995, all incorporated herein by reference).

Methods of Detecting Apoptotic Pathway Proteins

The present invention also provides methods for detecting or identifying apoptotic pathway proteins by contacting a sample with a BAG polypeptide or a functional fragment thereof and detecting the formation of complex between the BAG polypeptide or functional fragment thereof and the apoptotic pathway protein. The sample can be any composition suspected of containing an apoptotic pathway protein, including but not limited to cell extracts, cDNA expression libraries and recombinant proteins. Preferably, plant cell extracts, expression libraries of plant cDNAs, or recombinant proteins of plant genes are used. The BAG polypeptide or its functional fragment in the binding reaction can be a native polypeptide or a recombinant polypeptide protein. Preferably, the BAG polypeptide or its functional fragment is covalently bound to a detectable moiety, such as a reporter molecule, a radionuclide, or a polypeptide that facilitates the purification of the polypeptide, such as MBP and His tag. In certain embodiments, either before or after contacting with a sample containing an apoptotic pathway protein, a BAG polypeptide or its functional fragment is attached to a solid support via, e.g., its specific antibodies, or the polypeptide fused to it. One of ordinary skill in the art would know and further optimize the conditions that facilitate the interaction and formation of the complex between a BAG polypeptide or its functional fragment and an apoptotic pathway protein. The invention also includes the method of detecting or identifying BAG-associated apoptotic pathway protein where the interaction between the BAG polypeptide and the apoptotic pathway proteins occurs in vivo. The resulting complex of BAG polypeptide and apoptotic pathway proteins is then purified and its components other than the BAG polypeptide identified by any known protein purification and detection method. Several specific embodiments are described in more detail below.

1. In vitro Protein-Protein Interaction

Any in vitro protein-protein interaction assays can be used to detect an apoptotic pathway protein associated with a BAG polypeptide. In one example, a fusion protein is constructed comprising a BAG polypeptide or a functional fragment thereof and a tag peptide sequence (e.g., GST). The GST-fusion protein is then purified using glutathione-Sepharose beads (see, Kaelin et al., Cell 64:521, 1991). The bead-bound, purified fusion protein is then incubated with a sample, such as a plant cell extract (usually matabolically radio-labelled). Proteins that are not bound to the beads are washed away and those bound eluted. The bound proteins may be further fractionated by gel electrophoresis and detected by protein dyes or the radioactive label on the protein. They may be then used for raising antibodies, amino acid sequence analysis, and other in vitro analyses. Clones encoding the bound proteins may be isolated by any standard methods, including immunoscreening of an expression library, probe hybridization where the probe is based on partial amino acid sequences of the bound proteins.

In another example, a BAG polypeptide (or a functional fragment thereof) is first immobilized to a solid support (e.g., an ELISA plate). The immobilized BAG polypeptide is then mixed with a BAG binding molecule under conditions that allows the BAG binding molecule to interact with the immobilized BAG polypeptide to form a binding complex. Preferably, the BAG binding molecule is covalently bound to a detectable moiety, such as an enzyme that converts a colorless substrate into a colored product, which allows monitoring of the interaction between the BAG polypeptide and its binding molecule. The binding complex is then incubated with a sample that may contain a protein that competitively binds to the BAG polypeptide. Such competitive binding may be detected by measuring the dissociation of the BAG binding molecule via its detectable moiety.

2. Interaction Cloning

Any cloning methods that involve protein-protein interactions can be used for detecting or identifying BAG-associated apoptotic pathway proteins. For example, a recombinant BAG protein of the invention may be purified and radioactively labeled. Preferably, the BAG protein has a polypeptide tag fused to it to facilitate its purification. A cDNA expression library that contains a BAG-associated apoptotic pathway protein is plated and transferred onto nitrocellulose membranes. The membranes are then probed with radioactively labeled, purified recombinant BAG protein, and washed. Clones that interact with the BAG protein can be identified by radiography. The cDNA insert encoding a BAG-associated protein is then sequenced, and the amino acid sequence of the protein is deduced.

3. Coimmunoprecipitation

Coimmunoprecipitation assays are well known and can also be used to detect apoptotic pathway proteins that associate with a BAG polypeptide or its functional fragment of this invention. Briefly, a BAG polypeptide or its functional fragment is incubated with a sample that contains an apoptotic pathway protein. The mixture may be precleared with normal antiserum and protein A-Sepharose before immuoprecipitation. Immunoprecipitation can be performed by several methods such as by incubating protein A-Sepharose beads preadsorbed with anti-BAG antibody and precipitating the beads. After extensive washing, beads are boiled in Laemmli buffer and the eluted proteins are subjected to SDS-PAGE analysis. The proteins on the SDS-PAGE may be detected by dyes specific to proteins or by labels on the proteins if the proteins in the sample are labeled before incubation with the BAG polypeptide.

4. Two-Hybrid System

Alternatively, apoptotic pathway proteins may be identified by a yeast two-hybrid screening system. Briefly, in a yeast two-hybrid system, a fusion of nucleic acids encoding a DNA-binding domain of a transcription factor and a BAG polypeptide or functional fragment thereof (e.g., GAL4 DNA binding domain-BAG fusion) is constructed and transformed into a yeast cell containing a GAL binding site linked to a selectable marker gene. A library of cDNAs derived from an organism that contains BAG-associated apoptotic pathway proteins and fused to GAL4 activation domain is also constructed and co-transformed. When the cDNA in cDNA-GAL 4 activation domain fusion encodes a polypeptide that interacts with the BAG or its functional fragment, the selectable marker is expressed. Cells containing the cDNA are then grown, the construct isolated and characterized. The interaction between the polypeptides encoded by the isolated clones and the BAG polypeptide or BAG fragment may be verified by in vitro protein-protein interaction assays or communoprecipitation described supra.

5. Chromotography

Proteins that interact with a BAG polypeptide or functional fragment thereof of this invention may also be identified or detected by various chromotography techniques. For instance, after incubation of a BAG polypeptide or functional fragment with a sample that contains BAG-associated proteins, the mixture may be fractionated on various columns with a salt gradient or the like. In certain embodiments, cell extracts may be directly applied to chromotographic columns if the extracts contain an endogenous BAG polypeptide. The fractions that contain the BAG polypeptide or functional fragment thereof can be identified using, e.g., BAG antibodies, and further characterized by SDS-PAGE analysis. The polypeptides other than the BAG polypeptide or BAG fragment in those fractions may be further purified and sequences. The interaction between the identified polypeptides and the BAG polypeptide or BAG fragment may be verified by any other known protein-protein interaction assays.

The participation of any polypeptide that associates with a BAG polypeptide or functional fragment thereof in apoptosis may be tested in any systems where modulation of apoptosis may be assayed (see, e.g., Takayama et al., Cell 80:279-84, 1995; Asai et al., Plant Cell 12:1823-35, 2000). For example, a vector that is able to direct the expression of the nucleic acid encoding the BAG associated protein polypeptide is transfected in animal cell lines such as Jurkat T cell lines and 3T3 fibroblasts. Transfected cells with an elevated level of the protein can be detected by its antibodies. Such cells are then subject to several stimuli to induce apoptosis, such as anti-Fas and staurosporine. The survival rate of these cells is compared with that of cells transfected with control vectors that do not direct the expression of the BAG-associated protein to detect any apoptotic effect of the protein. In certain embodiments, the above transfected cells may be further transfected with a vector that directs the expression of the associated BAG polypeptide or fragment.

Alternatively, established plant programmed cell death model systems may also be used (see, e.g., Asai et al., Plant Cell 12:1823-35, 2000). For instance, protoplasts isolated from Arabidopsis leaves are transformed with a vector containing the nucleic acid encoding a BAG-associated protein, preferably fused to a reporter gene such as GFP. Protoplasts expressing the nucleic acid protein are then isolated or sorted by any technique applicable to the polypeptide encoded by the reporter gene. Fumonisin B1 (FB1) is added to such protoplasts and their viability after various incubation time is measured by using an Evan blue staining assay. Evan blue dye is excluded from viable cells and early stage apoptotic cells that retain intact plasma membranes. The effect of the BAG-associated polypeptide may be indicated by any differences in viability between protoplasts transformed with a vector containing the nucleic acid encoding the BAG-associated polypeptide and those transformed with a control vector.

Methods for Screening for Compounds that Modulate the Binding Between BAG Polypeptides and Bag Binding Proteins

The present invention also provides methods for screening for compounds that modulate (increase, decrease, or disrupt) the binding between BAG polypeptides (or functional fragments thereof) and BAG binding proteins. In certain preferred embodiments, such compounds are small molecules. In other preferred embodiments, such compounds are polypeptides or fragments thereof. Candidate compounds may be isolated or procured from a variety of sources, such as bacteria, fungi, plants, parasites, libraries of chemicals, random peptides or the like.

In certain embodiments, the present method compares the specific binding between a BAG polypeptide and a BAG binding protein in the absence of a candidate compound with that in the presence of the candidate compound. The increase of the specific binding in the presence of the compound indicates that the compound is able to facilitate the interaction between the BAG polypeptide and the BAG binding protein, while the decrease of the specific binding in the presence of the compound indicates that the compound is able to interfere with the interaction between the BAG polypeptide and the BAG binding protein.

In some related embodiments, the present method detects the dissociation of either a BAG polypeptide or a BAG binding protein from a binding complex between the BAG polypeptide and the BAG binding protein in the presence of a candidate compound. Such a dissociation of the BAG polypeptide or the BAG binding protein from the binding complex indicates that the compound is capable of disrupting the binding complex between the BAG polypeptide and the BAG binding protein.

The characterization of the binding between a BAG polypeptide and a BAG binding protein may be performed by any of the techniques known in the art that detect and/or characterize protein-protein interactions. Exemplary techniques are described above in relation to methods of detecting apoptotic pathway proteins. In certain preferred embodiments, the BAG polypeptide or its functional fragment is covalently bound to a detectable moiety, such as a reporter molecule and a raionuclide. Also preferred is that the BAG polypeptide or the BAG binding protein is attached to a solid support either before or after it forms a binding complex with its binding partner (i.e., the BAG binding protein for the BAG polypeptide or the BAG polypeptide for the BAG binding protein).

Transgenic Plants

As noted above, this invention also provides transgenic plants or plant cells with an increased or decreased expression level of a nucleic acid encoding a BAG polypeptide or functional fragments thereof.

A. General Methods of Generating Transgenic Plants or Transgenic Plant Cells

Generally, a transgenic plant is generated by (a) transforming plant cell with a nucleic acid of interest, (b) regenerating the plant cells to provide a differentiated plant, and (c) identifying a transformed plant that expresses the nucleic acid of interest. The nucleic acid of interest is usually contained in a vector. However, naked nucleic acid of interest may also be used even though only low efficiency transformation will likely occur.

1. Vectors

Although a general discussion of vectors of this invention is provided supra, the following description contains additional information specific to vectors used in plant cells. Usually, to be effective in regulating the expression level of a BAG gene in plant cells, a BAG-encoding nucleic acid in a vector is either operably linked, or linked in the antisense orientation, with a promoter functional in the plant cells. Additionally, a polyadenylation sequence or transcription control sequence, also recognized in plant cells, may be employed. It is preferred that the vector also contains one or more selectable marker genes so that the transformed cells can easily be selected from non-transformed cells in culture, as described herein.

General descriptions of plant expression vectors can be found in Gruber et al., “Vectors for Plant Transformation, in Methods in Plant Molecular Biology & Biotechnology” in Glich et al., Eds. pp. 89-119, CRC Press, 1993. Moreover, GUS expression vectors and GUS gene cassettes are available from Clontech Laboratones, Inc. (Palo Alto, Calif.) while GFP expression vectors and GFP gene cassettes are available from Aurora Biosciences (San Diego, Calif.).

a. Promoters

Any promoters functional in plant cells may be used for generating transgenic plants of this invention, including constitutive, inducible/developmentally regulated, and tissue specific promoters. Although endogenous promoters of BAG genes may be utilized for their transcriptional regulation, preferably, the promoters are foreign regulatory sequences. Such regulatory sequences may be obtained from plants, viruses or other sources.

Examples of constitutive promoters include, but are not limited to, the 35S RNA and 19S RNA promoters of cauliflower mosaic virus (CaMV), promoter for the coat protein promoter to TMV (Akamatsu et al., EMBO J. 6:307, 1987), promoters of seed storage protein genes such as Zma10Kz or Zmag12 (maize zein and glutelin genes, respectively), “housekeeping genes” that express in all cells such as Zmaact, a maize actin gene (see, Benfey et al., Science 244:174-181, 1989; Elliston in Plant Biotechnology, eds. Kung and Arntzen, Butterworth Publishers, Boston, Mass., p. 115-139, 1989), the patatin gene promoter from potato (see, e.g., Wenzler et al, Plant Mol. Biol. 12:41-45, 1989), the ubiquitin promoter (see, EP Patent Application 0342926), and the Chlorella virus DNA methyltransferase promoter (see, U.S. Pat. No. 5,563,328).

Inducible promoters are also useful within the context of the present invention. An inducible promoter is a promoter capable of directly or indirectly activating transcription of a nucleic acid sequence in response to an inducer. The inducer may be a biotic or abiotic insult such as a protein, a metabolite (sugar, alcohol, etc.), a growth regulator, a herbicide, or a phenolic compound, or a physiological stress imposed directly by heat, salt, toxic elements, etc., or indirectly through the action of a pathogen or disease agent such as a virus. A plant cell containing an inducible promoter may be exposed to an inducer by externally applying the inducer to the cell such as by spraying, watering, heating, exposure to light, exposure to a pathogen, or similar methods.

To be most useful, an inducible promoter preferably provides low or no expression in the absence of the inducer; provides high expression in the presence of the inducer; and uses an induction scheme that does not interfere with the normal physiology of the plant and has little effect on the expression of other genes. Examples of inducible promoters useful within the context of the present invention include those induced by chemical means, such as the yeast metallothionein promoter activated by copper ions (Mett et al., Proc. Natl. Acad. Sci. U.S.A. 90:4567, 1993); In2-1 and In2-2 regulator sequences activated by substituted benzenesulfonamides, e.g., herbicide safeners (Hershey et al., Plant Mol. Biol 17:679, 1991); the promoter sequence isolated from a 27 kD subunit of the maize glutathione-S-transferase (GST II) gene induced by N,N-diallyl-2,2-dichloroacetamide (common name: dichloramid) or benzyl-2-chloro-4-(trifluoromethyl)-5-thiazolecarboxylate (common name: flurazole) (PCT Publication No. WO 90/08830) the GRE regulatory sequences induced by glucocorticoids (Schena et al., Proc. Natl. Acad Sci. USA. 88:10421, 1991; (Aoyama and Chua, The Plant J. 11:605-612, 1997), and the alcohol dehydrogenase promoter induced by ethanol (Nagao et al. Miflin, Ed, Oxford Surveys of Plant Molecular and Cell Biology, Vol. 3, p. 384-438, Oxford University Press, Oxford, 1986). Other inducible promoters include those induced by pathogen attack described in PCT Publication Nos. WO 98/22599, WO99/504428 and U.S. Pat. No. 6,100,451, a chalcone synthase promoter, and the defense activated promoter (prop1-1) (Strittmatter et al., Bio/Technology 13:1085-1089, 1995). Inducible promoters have also been described in published Application No. EP89/103888.7 by Syngenta (formerly Ciba-Geigy), wherein a number of inducible promoters are identified, including the PR protein genes, especially the tobacco PR protein genes, such as PR-1a, PR-1b, PR-1c, PR-1, PR-A, PR-S, the cucumber chitinase gene, and the acidic and basic tobacco beta-1,3-glucanase genes. Wound inducible (WIN) promoters may also be useful in the context of the present invention, see Lindsey and Jones in: Plant Cell Line Selection, pp. 317-335, VCH Weinham, Germany, 1990, for a discussion thereon.

Tissue-specific promoters are another type of promoters that may be utilized in the present invention. Specific examples of tissue-specific promoter include, but are not limited to, shoot meristem-specific promoter described in Atanassova et al., Plant J. 2:291, 1992; tuber-directed class I patatin promoter described in Bevan et al., Nucleic Acids Res. 14:4625-38, 1986; promoters associated with potato tuber ADPGPP genes, Muller et al., Mol. Gen. Genet. 224:136-46, 1990; seed-directed promoter of β-conglycinin, also known as the 7S protein described in Bray, Planta 172:364-370, 1987; seed-directed promoters from maize zein genes described in Pedersen et al., Cell 29:1015-26, 1982; pollen-specific promoters such as those disclosed in U.S. Pat. No. 5,086,169 to Mascarenhas, U.S. Pat. No. 5,412,085 to Allen et al., and Plant 8:55-63, 1995; anther-specific promoter described in U.S. Pat. No. 5,477,002 to Tuttle et al.; and tapetum-specific promoter described in U.S. Pat. No. 5,470,359 to Huffman et al. and in WO 92/11379 to Draper et al.

It may also be desirable to include intron sequences in the promoter constructs since the inclusion of intron sequences in the coding region may result in enhanced expression and specificity. Thus, it may be advantageous to join the DNA sequences to be expressed to a promoter sequence that contains the first intron and exon sequences of a polypeptide which is unique to cells/tissues of a plant. Additionally, regions of one promoter may be joined to regions from a different promoter in order to obtain a chimeric promoter.

b. Markers

The vectors of the present invention, also preferably include at least one selectable or scorable marker/reporter that is functional in plant cells. A selectable marker gene includes any gene that confers a phenotype or trait on the host cells that allows transformed cells to be identified and selectively grown. Accordingly, the selection marker genes may encode polypeptides that confer on plant cells resistance to a chemical agent or to physiological stress, or a distinguishable phenotypic characteristic to the cells such that plant cells transformed with the recombinant nucleic acid molecule may be easily selected using a selective agent. Specific examples for the genes suitable for this purpose have been identified may be found in, e.g., Fraley, in Plant Biotechnology, eds. Kung and Amtzen, Butterworth Publishers, Boston, Mass., p. 395-407, 1989, and in Weising et al., Ann. Rev. Genet. 22:421-77, 1988.

2. Transformation

The transformation of plants in accordance with the invention may be carried out in essentially any of the various ways known to those skilled in the art of plant molecular biology (see, for example, Methods of Enzymology, Vol. 153, 1987, Wu and Grossman, Eds., Academic Press, incorporated herein by reference). As used herein, the term “transformation” means alteration of the genotype of a host plant or a host plant cell by the introduction of a heterologous nucleic acid sequence.

Methods of introducing vectors into plant cells or plant tissues may include physical and/or chemical means (e.g., electroportation, microinjection of plant cell protoplasts, particle bombardment, viral and bacterial infection/co-cultivation) and are applicable to both monocotyledenous and dicotyledenous plants. Potrykus, Annu. Rev. Plant Physiol., Plant Mol. Biol. 42:205-225, 1991, and Shimamoto et al., Nature 338:274-276, 1989. The principle methods of causing stable integration of exogenous DNA into plant genomic DNA include the following approaches: (1) Agrobacterium—mediated gene transfer; Horsch et al., Science 227:1229, 1985; Klee et al., Annu. Rev. Plant Physiol. 38:467-486, 1987; Klee et al., Mol. Bio. Of Plant Nucl. Genes 6:2-25, 1989; Gatenby, Plant Biotech., 93-112, 1989; White, Plant Biotech., 3-34 1989; (2) direct DNA uptake, Paszkowski et al, Mol. Bio. of Plant Nucl. Genes 6:52-68, 1989, including methods for direct uptake of DNA into protoplasts, Toriyama et. al., Bio/Technology 6:1072-1074, 1988; DNA uptake induced by brief electric shock of plant cells, Zhang et al, Plant Cell Rep. 7:379-384, 1988, and Fromm et al., Nature 319:791-792, 1986; DNA injection into plant cells or tissues by particle bombardment, Klein et al., Progress in Plant Cellular and Molecular Biology, 56-66, 1988, Klein et al., Bio/Technology 6:559-563, 1988, McCabe et al., Bio/Technology 6:923-926, 1988, and Sanford, Physiol. Plant 79:206-209, 1990; by the use of micropipette systems, Hess, Int. Rev. Cytol 107:367-395, 1987, Neuhaus et al., Theor. Appl Genet. 75:30-36, 1987, Neuhaus and Spangenberg, Physiol. Plant. 79:213-217, 1990, or by the direct incubation of DNA with germinating pollen, DeWet et al., Experimental Manipulation of Ovule Tissue, 197-209, 1985, Ohta, Y. Proc. Natl. Acad. Sci USA 83:715-719, 1986; or (3) the use of plant virus as gene vectors, Klee et al., Ann. Rev. Plant Physiol. 38:467-486, 1987; Futterer et al., Physiol. Plant 79:154-157, 1990.

Various plant cells, tissues and organs can serve as targets for Agrobacterium mediated transformation. For members of the Brassicaceae, they include thin cell layers (Charest et al., Theor. Appl. Genet. 75:438-444, 1988), hypocotyls (DeBlock et al., Plant Physiol. 91:694-701, 1989), leaf discs (Feldman, Plant Sci. 47:63-69, 1986), stems (Fry et al., Plant Cell Repts. 6:321-325, 1987), cotyledons (Moloney et al., Plant Cell Repts 8:238-242, 1989) and embryoids (Neuhaus et al., Theor. Appl. Genet. 75:30-36, 1987). One skilled in the art understands, however, that it may be desirable in some crops to choose a different tissue or method of transformation.

It may be useful to generate a number of individual transformed plants with any recombinant construct in order to recover plants free from any position effects. In certain embodiments it may be preferable to select plants that contain one copy of the introduced heterologous nucleic acid molecule in order to minimize co-suppression.

In one embodiment, expression vectors are introduced into plant tissues using co-cultivation of plant cells with viruses. For example, plant RNA viral based systems can used, typically by inserting the structural gene of interest into the coat promoter regions of a suitable plant virus under the control of a promoter. Plant RNA viral based systems are described, for example, in U.S. Pat. Nos. 5,500,360; 5,316,931, and 5,589,367, each of which is incorporated herein by reference in its entirety.

In other embodiments, the Agrobacterium Ti plasmid system is utilized, Watson et al., Recombinant DNA, a Short Course, Scientific American Books, 164-175, 1983. The tumor-inducing (Ti) plasmids of A. tumefaciens contain a segment of plasmid DNA known as transforming DNA (T-DNA) that is transferred to plant cells where it integrates into the plant host genome. The construction of the transformation vector system has two basic steps. First, a plasmid vector is constructed which replicates in E. coli. This plasmid contains the DNA encoding the protein of interest flanked by T-DNA border sequences that define the points at which the DNA integrates into the plant genome. Usually a gene encoding a selectable marker (such as a gene encoding resistance to an antibiotic such as Kanamycin) is also inserted between the left border (LB) and right border (RB) sequences; the expression of this gene in transformed plant cells gives a positive selection method to identify those plants or plant cells which have an integrated T-DNA region, Watson et al., Recombinant DNA, a Short Course, Scientific American Books, 164-175, 1983; White, Plant Biotechnology, 3-34, 1989. The second step entails transfer of the plasmid from E. coli to Agrobacterium. This can be accomplished via a conjugation mating system, or by direct uptake of plasmid DNA by Agrobacterium. For subsequent transfer of the T-DNA to plants, the Agrobacterium strain utilized contains a virulence (vir) genes for T-DNA transfer to plant cells (see, White, Plant Biotechnology, 3-34, 1989; Fraley, Plant Biotechnology, 395-407, 1989; Hajdukiewicz et al., Plant Mol. Bio. 25:989-994, 1994). Those skilled in the art recognize that there are multiple choices of Agrobacterium strains and plasmid construction strategies that can be used to optimize genetic transformation of plants. Methods of inoculation of the plant tissue vary depending upon the plant species and the Agrobacterium delivery system. A very convenient approach is the leaf disc procedure that can be performed with any tissue explant that provides a good source for initiation of whole plant differentiation. The addition of nurse tissue may be desirable under certain conditions. Other procedures such as the in vitro transformation of regenerating protoplasts with A. tumefaciens may be followed to obtain transformed plant cells as well, Potrykus, Plant Mol. Biol. 42:205-225, 1991; White, Plant Biotechnology, 3-34, 1989.

In certain embodiments, heterologous nucleic acid sequences can be introduced into a plant cell by contacting the plant cell using direct physical or chemical means. For example, the nucleic acid can be physically transferred by microinjection directly into plant cells by use of micropipettes or particle bombardment. Alternatively, the nucleic acid may be transferred into the plant cell by using polyethylene glycol which forms a precipitation complex with genetic material that is taken up by the cell (Paszkowske, et al., Proc. Natl Acad. Sci., USA 82:5824, 1985).

Another method for introducing nucleic acid into a plant cell is high velocity ballistic penetration by small particles with the nucleic acid to be introduced contained either within the matrix of small beads or particles, or on the surface thereof (Klein et al., Nature 327:70, 1987; U.S. Pat. Nos. 4,945,050, 5,036,006, and 5,100,792). Typically, when utilizing small particle bombardment, the DNA is adsorbed on microprojectiles such as magnesium sulfate crystals or tungsten particles, and the microprojectiles are physically accelerated into cells or plant tissues.

Heterologous nucleic acid can also be introduced into plant cells by electroporation (Fromm et al., Proc. Natl. Acad Sci., U.S.A. 82:5824, 1985). In this technique, plant protoplasts are electroporated in the presence of vectors or nucleic acids containing the relevant nucleic acid sequences. Electrical impulses of high field strength reversibly permeabilize membranes allowing the introduction of nucleic acids. Electroporated plant protoplasts reform the cell wall, divide and form plant callus. Selection of the transformed plant cells with the transformed gene can be accomplished using phenotypic markers as described herein.

After selecting the transformed cells, one can confirm expression of the introduced BAG gene. Simple detection of mRNA encoded by the inserted DNA can be achieved by well-known methods in the art, such as Northern blot analysis. The inserted sequence can be identified using the polyerase chain reaction and Southern blot analysis. The alteration in amount of BAG polypeptide in the transformed cells may be detected by western blot analysis using anti-BAG antibodies.

3. Regeneration of Transgenic plants

Transformed cells that express a nucleic acid encoding a BAG polypeptide or functional fragments is regenerated into a whole plant using any known techniques. The term “regeneration”, as used herein, refers to growing a whole plant from a protoplast, a plant cell, a group of plant cells (e.g., plant callus), a plant tissue, or a plant organ or part.

Regeneration from protoplasts varies from species to species of plants, but generally a suspension of protoplasts is first made. In certain species, embryo formation can then be induced from the protoplast suspension, to the stage of ripening and germination as natural embryos. The culture media will generally contain various amino acids and hormones, necessary for growth and regeneration. Examples of hormones utilized include auxin and cytokinins. It is sometimes advantageous to add glutamic acid and proline to the medium, especially for such species as corn and alfalfa. Efficient regeneration will depend on the medium, on the genotype, and on the history of the culture. If these variables are controlled, regeneration is reproducible.

Regeneration also occurs from plant callus, tissues, organs or parts. Transformation can be performed in the context of organ or plant part regeneration (see Methods in Enzymology, Vol. 118 and Klee et al., Ann. Rev. Plant Phys. 38:467, 1987). Utilizing the leaf disk-transformation-regeneration method of Horsch et al., Science 227:1229, 1985, disks are cultured on selective media, followed by shoot formation in about 2-4 weeks. Shoots that develop are excised from calli and transplanted to appropriate root-inducing selective medium. Appropriate selection media are known in the art and described by Curry and Cassells in: Plant Cell Culture Protocols, pp. 31-43, Humana Press, Totowa, N.J., 1999; Blackwell et al, IBID 19-30, 1999; Franklin and Dixon in: Plant Cell Culture, pp. 1-25, IRL Press, Oxford, 1994. Rooted plantlets are transplanted to soil as soon as possible after roots appear. The plantlets can be repotted as required, until reaching maturity.

In vegetatively propagated crops, the mature transgenic plants are propagated by the taking of cuttings or by tissue culture techniques to produce multiple identical plants. Selection of desirable transgenotes is made and new varieties are obtained and propagated vegetatively for commercial use. In seed propagated crops, the mature transgenic plants can be self crossed to produce a homozygous inbred plant. The inbred plant produces seed containing the newly introduced heterologous nucleic acid sequence(s). These seeds can be grown to produce plants that would produce the selected phenotype, e.g., pathogen resistance.

Parts obtained from the regenerated plant, such as flowers, seeds, leaves, branches, fruit, and the like are included in the invention, provided that these parts comprise cells that have been transformed as described. Progeny and variants, and mutants of the regenerated plants are also included within the scope of the present invention, provided that these parts comprise the introduced heterologous nucleic acid sequences.

B. Generation of Plants and Plant Cells with Desirable Traits

The present invention is directed to the identification of genes encoding plant homologues of animal BAG-1 protein. In animals, BAG-1 gene, when overexpressed, either alone or in combination with a Bcl-2 gene, blocks or delays apoptosis (Takayama et al., Cell 80:279-84). In addition, there is evidence indicating that heterologous apopototic pathway proteins function in plants and that expression of animal anti-apoptosis genes renders the plants resistance to biotic and abiotic insults, and inhibition of senescence. Thus, over-expression of nucleic acids encoding plant BAG polypeptides or functional fragments thereof would provide plants with similar or even more pronounced beneficial features. Accordingly, the invention provides transgenic plants or plant cells that contain and express transgenes comprising nucleic acids encoding BAG polypeptides or functional fragments thereof having substantial sequence identity with A. thaliana BAG polypeptides operably linked to promoters.

Biotic insults are insults incurred by a plant as the direct or indirect result of a challenge by a biotic agent. Biotic agents include, for example, insects, fungi, bacteria, viruses, nematodes, viroids, mycloplasmas, etc. Biotic agents typically induce programmed cell death in affected plant cells. Such programmed cell death is thought to occur to inhibit the spread of an invading pathogen. However, in one embodiment of the present invention, nucleic acid sequences encoding BAG polypeptides or functional fragments thereof are delivered to plant cells and the plants that develop therefrom have a demonstrated resistance to a variety of biotic agents.

Leading biotic agents include various pathogens such as fungi and viruses. An exemplary pathogen is the fungal pathogen Sclerotinia sclerotiorum, which is one of the most nonspecific and omnivorous plant pathogens known (see, e.g., Purdy, Phytopathology 69:875-880, 1979). Further, a variety of other economically important pathogens are known including the fungi Botrytis cinerea, Magnaportyhe grisea, Phytophthora spp, Cochliobolus spp, Fusarium graminearum, and Fusarium spp, the Nemtodes Meloidogyne spp (Root knot nematodes), viruses such as tobacco mosaic virus (TMV) and tomato spotted wilt virus (TSWV), tobacco etch virus (TEV), tobacco necrosis virus (TNV), wheat streak mosaic virus (WSMV), soil borne wheat mosaic virus (SBWMV), barley yellow dwarf virus (BYDV), the bacteria Pseudomonas spp and Xanthomonas ssp., as well as many others. A number of examples are available and can be readily identified by those of skill in the art see, e.g., Plant Pathology, 4 th ed., Agrios, ed., Academic Press, San Diego, 1997.

Abiotic agents which cause abiotic insults include, for example, environmental factors such as low moisture (drought), high moisture (flooding), nutrient deficiency, radiation levels, air pollution (ozone, acid rain, sulfur dioxide, etc.), high temperature (hot extremes), low temperature (cold extremes), and soil toxicity, as well as herbicide damage, pesticide damage, or other agricultural practices (e.g., over-fertilization, improper use of chemical sprays, etc.). Accordingly, given that such abiotic agents play an increasing role in the viability of a variety of plant types including, food crops and ornamentals, the present invention can be utilized to produce plants with increased resistance to these insults.

One skilled in the art will readily recognize that given the disclosure provided herein, resistance to a particular biotic or abiotic insult/agent can be easily tested using whole plant or leaf sections as appropriate for the action of the particular agent. For example, a plant leaf may be inoculated with virus and lesion development and expansion may be measured at different time intervals. Another example may be that whole plants are subject to an abiotic insult such as high temperature and stress responses to the insult or survival rates of the plants may be measured.

Senescence in plants is known to be a regulated process ultimately resulting in cell death (see generally, Guiamet et al., Plant Cell Phys. 31.1123-1130, 1990 for a detailed discussion thereon). Further, it is accompanied by many of the biochemical and structural changes such as induction of cysteine proteases, RNases, etc., that are consistent with programmed cell death. Inhibiting senescence can lead to longer shelf-lives for vegetables and fruits, as well as leading to increase longevity and aesthetic appeal of cut flowers and other ornamentals. In addition, in the living plant increased flowering duration and fruit production may be achieved. Accordingly, the present invention has wide utility in both the food stuff market as well as the ornamental market.

Any known methods for measuring senescence in plants or plant cells may be used to screen for transgenic plants of this invention with a decreased level of senescence. Such methods include, for example, characterization of fruit ripening process, measurement of flower life, and detection of ethylene production (see, e.g., U.S. Pat. No. 5,702,933, Ryu et al., Proc. Natl. Acad. Sci. USA 94:12717-21, 1997).

Besides providing transgenic plants or plant cells with increased levels of BAG polypeptides, the invention also provides transgenic plants with decreased levels of BAG polypeptides. Such transgenic plants may contain and express transgenes comprising nucleic acids encoding BAG polypeptides or functional fragments thereof having substantial sequence identity with A. thaliana BAG polypeptides linked to a promoter in the antisense orientation. Alternatively, the decreased levels of BAG polypeptides may be achieved by screening transgenic plants or plant cells that contain and express transgenes comprinsing BAG-encoding nucleic acids operably linked to promoters for co-suppression. “Co-suppression” refers to suppression of expression of an endogenous gene when transgenic plants contain multiple copies of a transgene having a nucleic acid sequence similar or identical to the endogenous gene.

An additional approach for generating transgenic plants with decreased levels of BAG polypeptides is to screen for transgenic plants with functional BAG genes knocked out. Such transgenic plants may be obtained by screening transgenic plants containing randomly inserted heterogenous DNA (e.g., T-DNA) for the transgenic plants having insertions of heterogenous DNA within the coding region of a BAG gene. The technique for obtaining transgenic knockout plants is known (see, McKinney et al. Plant J. 8: 613-22, 1995; Filleur et al., FEBS Lett. 48:220-224, 2001; Craciun et al. FEBS Lett. 487:234-238, 2000). For instance, transgenic Arabidopsis plants containing random T-DNA insertions may be obtained from University of Wisconsin-Madison Biotechnology Center (http://www.biotech.wisc.edu/Arabidopsis), Arabidopsis Biological Resource Center at Columbus, Ohio (http:/aims.cps.msu.edu/aims) and Nottingham Arabidopsis Stock Center (http://nasc.nott.ac.uk). These transgenic plants may be screened for T-DNA insertions in the coding regions of BAG gene using PCR primers complementary to portions of T-DNA and a BAG gene, respectively. The resulting PCR product may be probed with the BAG gene or a fragment thereof to confirm that it contains a T-DNA insertion in the BAG gene.

Method of Modulating Apoptosis

The invention also provides a method of modulating apoptosis in a plant. Generally, the method comprises the following steps: (a) transforming cells of the plant with a nucleic acid molecule comprising a promoter functional in cells of the plant and operably linked to a nucleic acid sequence encoding a BAG polypeptide or functional fragments thereof, (b) regenerating the plant cells to provide a differentiated plant, and (c) identifying a transformed plant which expresses the coding sequence and exhibits altered apoptosis. Transformation of plant cells with a nucleic acid molecule, regeneration of plants from transformed plant cells, and identification of transformed plants that express the nucleic acid molecule may be carried out as described supra. Any known method for assaying apoptosis may be used to identify transformed plants with altered apoptosis (see, e.g., Mittler et al., Plant Cell 7:29-42, 1995; Mittler et al., Plant Mol Biol. 34:209-11, 1997). For instance, a transformed plant or a portion of the plant may be challenged with a biotic or abiotic agent and the morphology of the inoculation site is observed for apoptotic signs. These inoculation sites can be further characterized by subsequent analysis for DNA fragmentation (by agarose gel electrophoresis), nuclear condensation (by Hoechst or DAPI staining), the change of the number of TUNEL positive cells compared to control samples (TUNEL kits available from Oncor and Boehringer-Mannheim).

From the foregoing it will be appreciated that, although specific embodiments of the invention have been described herein for purposes of illustration, various modifications may be made without deviating from the spirit and scope of the invention. In addition, all references including patents, patent applications, and journal articles are incorporated herein in their entirety. Accordingly, the invention is not limited except as by the appended claims.

### EXAMPLES

### Example 1

### Isolation and Characterization of Plant BAG-1 cDNA

To identify plant molecules that are homologues of mammalian molecules of the cell death pathway, nucleic acid sequences were identified by higher algorithm computer searches. Four putative BAG homologues were identified using a profile-profile alignment method (Rychlewski et al., 2000 Protein Sci. 2:232-41). The sequences corresponded to four different accessions in the Gene Bank database, gi3068705, gi3702325, gi3157923 and gi3132472. The primers, 5′-GGGAGGTGAGACCTGGTGG-3′ (SEQ ID NO:9) and 5′-GGCCGAAGGCGAAGCTGGCGG-3′ (SEQ ID NO:10), were used to generate a PCR fragment from genomic A. thaliana DNA corresponding to gi3068705, and the PCR fragment was used to probe a cDNA library. The cDNA library CD4-7 was obtained from the Arabidopsis Biological Resource Center (Newman et al., 1994 Plant Physiol. 106:1241-55). A single full-length cDNA was initially identified, FIG. 1. The predicted amino acid sequence of A. thaliana BAG-1 is shown in FIG. 2.

### Example 2

### Isolation of A. thaliana HSP70

Mammalian BAG-1 associates with HSP/HSC70. To identify the plant homologue of HSP70, primers were made that were complementary to the published mammalian cDNA sequence. The primers were combined with a pool of A. thaliana cDNA and PCR amplification performed. The amplified PCR product was subcloned and sequenced. The nucleic acid sequence is identical to Genbank accession number x74604.

### Example 3

### In vitro Binding of A. thaliana BAG-1 to Mammalian BCL-2 and A. thaliana HSP70

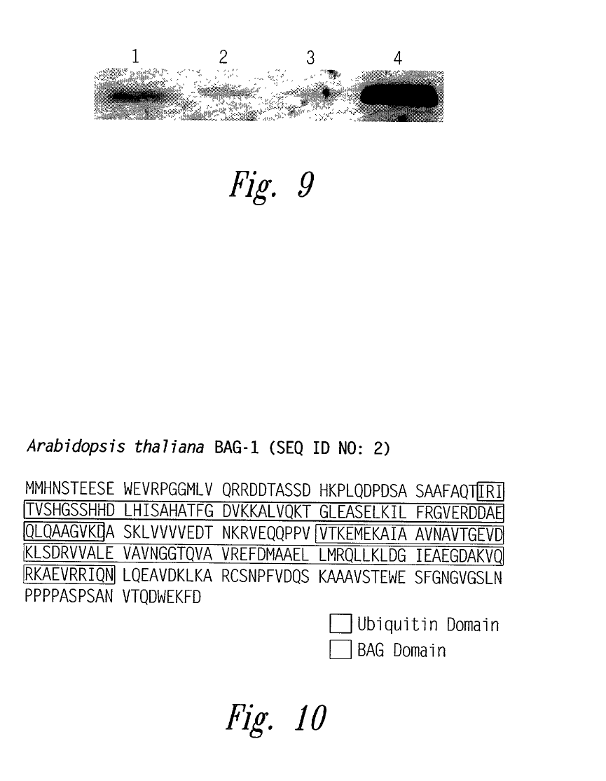

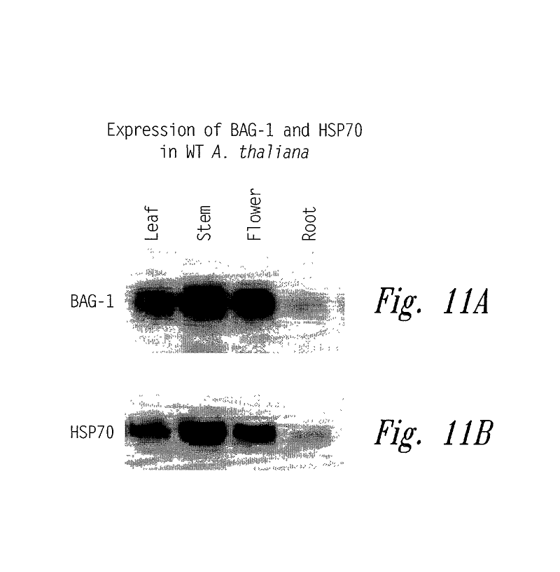

The cDNA sequence that encodes A. thaliana HSP70 was subcloned into a pGEX-X prokaryotic expression vector (Amersham Pharmacia) to produce a glutathione-S-transferase (GST)-HSP70 fusion protein in E. coli BL21 cells. After induction of protein expression, GST-HSP70 was isolated by affinity purification using glutathione-Sepharose according to standard protocol. A GST-Bcl-2 fusion was prepared in a similar manner using a human Bcl-2 cDNA. A. thaliana BAG-1 cDNA was cloned into a pZL1 vector (Gibco BRL) with a T7 promoter for expression. Radiolabeled (³⁵S-methionine) BAG-1 protein was generated directly from plasmid DNA using a TNT kit (Promega). In vitro binding was performed as described by Choi et al., 2000 Virology 267:185-98 or by Takayama et al., Cell 80:279-284, 1995. Results of this experiment are shown in FIG. 9.

### Example 4

### Northern Blot Analysis of BAG-1 Gene (SEQ ID NO: 1) in Arabidopsis Tissues

Total RNA was isolated from fresh tissue of Arabidopsis thaliana Col-1 using Trizol reagent (Gibco). Plant parts (leaves, stems, flower and roots) were ground in the presence of Trizol and the resulting liquid (approximately 1 ml) was transferred to an eppendorf tube and incubated at room temperature for 10 minutes. Two hundred microliters of chloroform was added to each tube and tubes were shaken for 15 seconds by hand then centrifuged at 11,600 RPM at 4° C. for 10 minutes. The supernatant was transferred to a new tube and 500 μl of isopropanol was added to precipitate the RNA. The tubes were mixed by inversion and incubated at room temperature for 10 minutes. The tubes were centrifuged at 11,600 RPM at 4° C. for ten minutes and the supernatant was discarded and the tubes inverted and pellets air dried for 30 minutes at room temperature. The pellets were resuspended in 25 μl of DEPC treated water and the concentration was determined spectrophotometrically. Ten μg of each sample were loaded on a 1.2% agarose gel containing 5% formaldehyde and electrophoresed at 150 volts for 3 hours. RNA was transferred directly to nylon membrane dried and probed with Arabidopsis BAG-1 cDNA fragment. Membranes were hybridized at 65° C. for 16 hours, and washed 1× with 40 mM NaHPO₄, 0.5% BSA, 1 mM EDTA, 5% SDS at room temperature and 2× with 40 mM NaHPO₄, 1 mM EDTA, 1% SDS at 65° C. Autoradiographs were exposed for 24 hours at −80° C.

### Example 5

### Detection of BAG Homologues in Tobacco

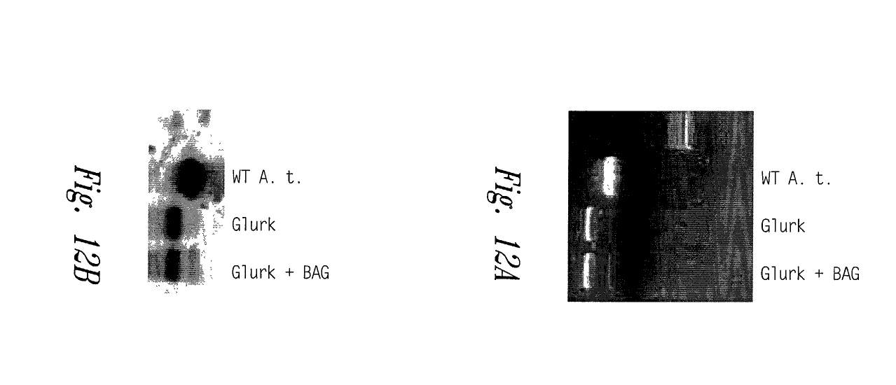

To determine whether a BAG homologue is present in tobacco plants, Southern blot of tobacco genomic DNA was performed using an Arabidopsis BAG clone as a probe. No hybridization was detected with tobacco genomic DNA while hybridization was detected with Arabidopsis genomic DNA as a positive control. However, using primers to Arabidopsis BAG-1 genomic clone (i.e., GenBank Accession No. 3068705), a PCR product was generated from tobacco (Glurk) genomic DNA (FIG. 12A, lane marked “Glurk”). A PCR product having the same size as that from Glurk genomic DNA was also detected from transgenic tobacco containing an Arabidopsis BAG gene (FIG. 12A, lane marked “Glurk +BAG”). The gel containing the above OPCR product as well as a PCR product from wild type Arabidopsis thaliana as a positive control was probed with an Arabidopsis BAG clone (FIG. 12B). The autoradiograph shows that the PCR product from Glurk hybridized with the Arabidopsis BAG clone and thus that a BAG homologue is present in tobacco plants.

We claim:

1. An isolated nucleic acid molecule comprising a nucleic acid sequence encoding a BAG polypeptide or functional fragments thereof, the polypeptide having at least 70% amino acid identity with SEQ ID NOS: 2, 4, 6, or 8.

2. The nucleic acid molecule of claim 1, wherein the BAG polypeptide is a plant BAG polypeptide.

3. The nucleic acid molecule of claim 2, wherein the BAG polypeptide is an Arabidopsis thaliana BAG polypeptide.

4. The nucleic acid molecule of claim 3, wherein the BAG polypeptide is an A. thaliana BAG-1 polypeptide presented in SEQ ID NO:2.

5. The nucleic acid molecule of claim 3, wherein the BAG polypeptide is an A. thaliana BAG-2 polypeptide presented in SEQ ID NO:4.

6. The nucleic acid molecule of claim 3, wherein the BAG polypeptide is an A. thaliana BAG-3 polypeptide presented in SEQ ID NO:6.

7. The nucleic acid molecule of claim 3, wherein the BAG polypeptide is an A. thaliana BAG-4 polypeptide presented in SEQ ID NO:8.

8. The nucleic acid molecule of claim 4 comprising SEQ ID NO:1.

9. The nucleic acid molecule of claim 5 comprising SEQ ID NO:3.

10. The nucleic acid molecule of claim 6 comprising SEQ ID NO:5.

11. The nucleic acid molecule of claim 7 comprising SEQ ID NO:7.

12. An isolated nucleic acid molecule comprising a nucleotide sequence encoding a functional fragment of a BAG polypeptide having at least 70% identity with amino acids 131-210, amino acids 138-219, or amino acids 131-219 of SEQ ID NO:2.

13. The nucleic acid molecule of claim 12 wherein the nucleotide sequence comprises nucleotides 496-735, nucleotides 517-762, or nucleotides 496-762 of SEQ ID NO:1.

14. The nucleic acid molecule of claim 12 wherein the functional fragment comprises amino acids 131-210, amino acids 138-219, or amino acids 131-219 of SEQ ID NO:2.

15. An isolated nucleic acid molecule comprising a nucleotide sequence encoding a functional fragment of a BAG polypeptide having at least 70% identity with amino acids 48-109 or amino acids 48-118 of SEQ ID NO:2.

16. The nucleic acid molecule of claim 15 wherein the nucleotide sequence comprises nucleotides 247-432 or nucleotides 247-459 of SEQ ID NO:1.

17. The nucleic acid molecule of claim 15 wherein the functional fragment comprises amino acids 48-109 or amino acids 48-118 of SEQ ID NO:2.

18. An isolated nucleic acid molecule comprising a polynucleotide sequence selected from the group consisting of:
a) SEQ ID NO:1
b) SEQ ID NO:3
c) SEQ ID NO:5
d) SEQ ID NO:7
e) a polynucleotide sequence that hybridizes to SEQ ID NOS: 1, 3, 5, or 7 under moderately stringent condition; and
f) a polynucleotide sequence that is a complement of any polynucleotide sequence of a-d.

19. A nucleic acid vector comprising the nucleic acid molecule of claim 1.

20. The vector of claim 19 comprising a nucleic acid molecule selected from the group presented in SEQ ID NO:1, SEQ ID NO:3, SEQ ID NO:5, and SEQ ID NO:7.

21. The vector of claim 19 comprising a nucleic acid molecule encoding an amino acid sequence selected from the group presented in SEQ ID NO:2, SEQ ID NO:4, SEQ ID NO:6, and SEQ ID NO:8.

22. The vector of claim 19 wherein the nucleic acid molecule is under the control of a constitutive or inducible promoter.

23. The vector of claim 19 comprising the nucleic acid molecule of claim 12.

24. The vector of claim 19 comprising the nucleic acid molecule of claim 14.

25. The vector of claim 19 comprising the nucleic acid molecule of claim 15.

26. The vector of claim 19 comprising the nucleic acid sequence of claim 17.

27. A host cell containing the nucleic acid vector of claim 19.

28. The cell of claim 27 wherein the cell is selected from the group consisting of a mammalian cell, a yeast cell, a plant cell, and a bacterial cell.

29. An isolated BAG polypeptide comprising a polypeptide that has at least 70% amino acid identity with SEQ ID NOS: 2, 4, 6, or 8, or a functional fragment of the isolated BAG polypeptide.

30. The BAG polypeptide or the functional fragment of claim 29 wherein the BAG polypeptide or the functional fragment associates with a Bcl-2 related protein.

31. The polypeptide of claim 29 wherein the BAG polypeptide is a plant BAG polypeptide.

32. The polypeptide of claim 30 wherein the BAG polypeptide is an A. thaliana BAG polypeptide.

33. The polypeptide of claim 31 wherein the BAG polypeptide is an A. thaliana BAG-1 polypeptide presented in SEQ ID NO:2.

34. The polypeptide of claim 31 wherein the BAG polypeptide is an A. thaliana BAG-2 polypeptide presented in SEQ ID NO:4.

35. The polypeptide of claim 31 wherein the BAG polypeptide is an A. thaliana BAG-3 polypeptide presented in SEQ ID NO:6.

36. The polypeptide of claim 31 wherein the BAG polypeptide is an A. thaliana BAG-4 polypeptide presented in SEQ ID NO:8.

37. The functional fragment of claim 29 comprising a polypeptide having at least 70% identity with amino acids 131-210, amino acids 138-219, or amino acids 131-219 of SEQ ID NO:2.

38. The functional fragment of claim 29 comprising a polypeptide having at least 70% identity with amino acids 48-109 or amino acids 48-118 of SEQ ID NO:2.

39. An antibody comprising an immunoglobulin or antigen-binding fragment thereof that specifically binds the BAG polypeptide or the functional fragment of claim 29.

40. The antibody of claim 39 wherein the BAG polypeptide is an A. thaliana BAG polypeptide.

41. The antibody of claim 39 wherein the BAG polypeptide is an A. thaliana BAG-1 polypeptide presented in SEQ ID NO:2.

42. The antibody of claim 39 wherein the BAG polypeptide is an A. thaliana BAG-2 polypeptide presented in SEQ ID NO:4.

43. The antibody of claim 39 wherein the BAG polypeptide is an A. thaliana BAG-3 polypeptide presented in SEQ ID NO:6.

44. The antibody of claim 39 wherein the BAG polypeptide is an A. thaliana BAG-4 polypeptide presented in SEQ ID NO:8.

45. The antibody of claim 39 wherein the functional fragment has an amino acid sequence comprising amino acids 131-210, amino acids 138-219, or amino acids 131-219 of SEQ ID NO:2.

46. The antibody of claim 39 wherein the functional fragment has an amino acid sequence comprising amino acids 48-109 or amino acids 48-118 of SEQ ID NO:2.

47. The antibody of claim 39 wherein the antibody modulates apoptosis.

48. The antibody of claim 39 wherein the antibody is a polyclonal, a monoclonal, a chimeric antibody, a humanized antibody, a single chain antibody, or an antigen-binding fragment.

49. A method of detecting an apoptotic pathway protein comprising: contacting a sample with the BAG polypeptide or functional fragment thereof of claim 29 under conditions that permit formation of a complex between the BAG polypepetide or functional fragment thereof and the apoptotic pathway protein, and detecting the complex and the apoptotic pathway protein in the complex.

50. The method of claim 49 wherein the BAG polypeptide is covalently bound to a detectable moiety.

51. The method of claim 50 wherein the detectable moiety is a reporter molecule.

52. The method of claim 50 wherein the detectable moiety is a radionuclide.

53. The method of claim 49 wherein the apoptotic pathway protein is selected from the group consisting of a caspase, a rev-caspase, Bcl-2, Bcl-2 family members, Apaf-1, Bad, Bax, Ced-9, Ced-4, and HSP70.

54. The method of claim 49, wherein the sample comprises a cDNA expression library.

55. A nucleic acid molecule comprising the nucleic acid molecule of claim 1 and a nucleic acid molecule encoding the transcription activation domain or the DNA-binding domain of a transcription factor.

56. A method of identifying an apoptotic pathway protein with a yeast two-hybrid screening system comprising transforming a yeast cell with a vector comprising the nucleic acid molecule of claim 55.

57. A method of modulating apoptosis in a plant comprising:
transforming cells of the plant with a nucleic acid molecule comprising a promoter functional in cells of the plant and operably linked to a nucleic acid sequence encoding the BAG polypeptide or the functional fragment of claim 29, regenerating the plant cells to provide a differentiated plant, and identifying a transformed plant which expresses the coding sequence and exhibits altered apoptosis.

58. The method of claim 57 wherein the promoter is an inducible promoter.

59. The method of claim 58 wherein the promoter is inducible by a plant pathogen.

60. The method of claim 57 wherein the promoter is a constitutive promoter.

61. The method of claim 57 wherein the promoter is a tissue-specific promoter.

62. A transgenic plant or transgenic plant cell which contains and expresses a transgene comprising any one of the nucleic acid of claim 1, 12, 15, or 18 operably linked to a promoter.

63. The transgenic plant or transgenic plant cell of claim 62 selected from the group consisting of graminaceae, solanaceae, rosaceae, compositeae, leguminaceae, brassicaceae, and cucurbitaceae.

64. The transgenic plant or transgenic plant cell of claim 62 wherein the promoter is a plant inducible promoter.

65. The transgenic plant or transgenic plant cell of claim 64 wherein the promoter is inducible by a plant pathogen.

66. The transgenic plant or transgenic plant cell of claim 62 wherein the promoter is a tissue-specific promoter.

67. The transgenic plant or transgenic plant cell of claim 62 wherein the promoter is a constitutive promoter.

68. The transgenic plant or transgenic plant cell of claim 62 wherein the plant is biotic insult resistant.

69. The transgenic plant or transgenic plant cell of claim 68 wherein the biotic insult is induced by an insect.

70. The transgenic plant or transgenic plant cell of claim 68 wherein the biotic insult is induced by a plant pathogen.

71. The transgenic plant or transgenic plant cell of claim 70 wherein the pathogen is selected from the group consisting of a fungus, a nematode, a bacterium and a virus.

72. The transgenic plant or transgenic plant cell of claim 62 wherein the plant or plant cell is abiotic insult resistant.

73. The transgenic plant or transgenic plant cell of claim 72 wherein the abiotic insult is induced by an agent selected from the group consisting of high moisture, low moisture, salinity, nutrient deficiency, air pollution, high temperature, low temperature, soil toxicity, herbicides, and insecticides.

74. The transgenic plant of claim 62 wherein at least a portion of the plant exhibits a decreased level of senescence.

75. The transgenic plant cell of claim 62 selected from the group consisting of protoplasts, gamete producing cells, and cells capable of regenerating into a whole plant.

76. A transgenic plant or transgenic plant cell which contains and expresses a transgene comprising any one of the nucleic acid of claim 1, 12, 15 or 18 linked to a promoter in the antisense orientation.

77. The transgenic plant or transgenic plant cell of claim 76 selected from the group consisting of graminaceae, solanaceae, rosaceae, compositeae, leguminaceae, brassicaceae, and cucurbitaceae.

78. The transgenic plant or transgenic plant cell of claim 76 wherein the promoter is a plant inducible promoter.

79. The transgenic plant or transgenic plant cell of claim 78 wherein the promoter is inducible by a plant pathogen.

80. The transgenic plant or transgenic plant cell of claim 76 wherein the promoter is a tissue-specific promoter.

81. The transgenic plant or transgenic plant cell of claim 76 wherein the promoter is a constitutive promoter.

82. The transgenic plant cell of claim 76 selected from the group consisting of protoplasts, gamete producing cells, and cells capable of regenerating into a whole plant.

83. A method for screening for a compound that increases the specific binding between a BAG polypeptide and a BAG binding protein, comprising
(a) combining the BAG polypeptide with the BAG binding protein in the absence of a candidate compound under conditions that allow specific binding between the BAG polypeptide and the BAG binding protein;
(b) combining the BAG polypeptide with the BAG binding protein in the presence of the candidate compound under the conditions of step (a); and
(c) comparing the specific binding between the BAG polypeptide and the BAG binding protein of step (a) with that of step (b) to thereby determine whether the candidate compound is capable of increasing the specific binding between the BAG polypeptide and the BAG binding protein.

84. A method for screening for a compound that decreases the specific binding between a BAG polypeptide and a BAG binding protein, comprising
(a) combining the BAG polypeptide with the BAG binding protein in the absence of a candidate compound under conditions that allow specific binding between the BAG polypeptide and the BAG binding protein;
(b) combining the BAG polypeptide with the BAG binding protein in the presence of the candidate compound under the conditions of step (a); and
(c) comparing the specific binding between the BAG polypeptide and the BAG binding protein of step (a) with that of step (b) to thereby determine whether the candidate compound is capable of decreasing the specific binding between the BAG polypeptide and the BAG binding protein.

85. A method for screening for a compound that disrupts the binding between a BAG polypeptide and a BAG binding protein comprising
(a) contacting a candidate compound with a binding complex comprising the BAG polypeptide and the BAG binding protein; and
(b) detecting either the BAG polypeptide or the BAG binding protein that dissociates from the binding complex to thereby determine whether the candidate compound is capable of disrupts the binding between the BAG polypeptide and the BAG binding protein.

86. The method of any one of claims 83-85 wherein the BAG polypeptide is the isolated BAG polypeptide of claim 29.

87. The method of any one of claims 83-85 wherein either the BAG polypeptide or the BAG binding protein is covalently bound to a detectable moiety.

88. The method of claim 87 wherein the detectable moiety is a reporter molecule.

89. The method of claim 87 wherein the detectable moiety is radionuclide.


SEQUENCE LISTING (10 sequences)

SEQ ID NO: 1 - DNA, 1003 bp, Arabidopis thaliana
gtgcatcatccgtctatcaataccctgacaaggaaaaccctaattttttttggcccaaagggttttatactttaatctcatactccgattcattgtcgaaaaacgatgatgcataattcaaccgaagaatcggaatgggaggtgagacctggtggtatgctcgtccaacgaagagacgacaccgcttcctccgaccacaaacctctccaggatcccgactctgcttccgccgcttttgctcaaaccatcagaatcactgtttctcatggctcatcgcaccacgatcttcatatttctgctcacgccactttcggggatgtaaagaaagctcttgttcagaaaactggattggaagctagtgaattgaagatcttgttcagaggagttgagagagatgatgctgaacaattgcaagctgctggtgttaaggatgcgtctaagcttgttgttgttgttgaggatacgaataagagagtggaacaacagcctcctgtggtaactaaagagatggaaaaagctattgctgctgttaacgcggttacaggagaggtcgataagctctcggatagagttgttgctttagaagttgctgtgaatggagggacgcaagttgcggtgcgggagtttgacatggctgcagagcttcttatgaggcagctgctcaaattggatggcattgaggctgaaggggacgctaaagtacagcgtaaggctgaggtacgtagaatccaaaacttgcaggaggctgtggataagttgaaggcaagatgttcaaatccgtttgtggatcagagcaaagctgcagctgtaagcactgagtgggaatcgttcggaaacggtgtcggaagcttgaaccctcctccgccagcttcgccttcggccaatgtaactcaagattgggagaaatttgactgacattttagtactgctacgttgccttggaaaaaaatgcttattgaaactgtttatctttttgattattgttaaatattggaaaacgacc

SEQ ID NO: 2 - protein, 269 aa, Arabidopis thaliana
MMHNSTEESEWEVRPGGMLVQRRDDTASSDHKPLQDPDSASAAFAQTIRITVSHGSSHHDLHISAHATFGDVKKALVQKTGLEASELKILFRGVERDDAEQLQAAGVKDASKLVVVVEDTNKRVEQQPPVVTKEMEKAIAAVNAVTGEVDKLSDRVVALEVAVNGGTQVAVREFDMAAELLMRQLLKLDGIEAEGDAKVQRKAEVRRIQNLQEAVDKLKARCSNPFVDQSKAAAVSTEWESFGNGVGSLNPPPPASPSANVTQDWEKFD

SEQ ID NO: 3 - DNA, 3132 bp, Arabidopis thaliana
atgatgcctgtgtacatggatccatcacaaccatgtcaaatgagaccacaggaatactattatcaaggatttggtaataactctcagcatatggcaatggatgctccaccaccgtgtcatggaagctgcgttcacggcaactttcctgcttactggcctccttgttatcccccacaagtaccgtaccatcaatgctgcatgaatcgttccgctttccatcctcctcacgcgtcttacgctccgtcttgttacgttcatccaccatttccagttggttatcaaccttggtttgatgttgagaaggatgtgcctgggaagcatcactgcgggaaatgttcttcccagatgtgtgatttgaagaaagacagaggcgttgtgattgaagagcatgagcctgagattgagaaaggagaagctgtacttccagttcgttctactaactgcccttacccaattatatggattcctcatgagaatgctaggaatcaggaatatagaagctctcttgggttagggaagcataaccagcctcctgctgaagttagagctcctgacaatatgacgattcagaaaagttttcctgaatcttggcgtggttgttttccatttgatgagagcagcatgaagtcgttggtacagaatcaagacagtaaaaaggcgcagaatgggaaaaccgtggaagccccgtttgatatcagcaaattcaaatccttattgcaaggtcaagacatgaaggaagcgcaaatccagaagaacaaggaagagctgggacagcttacgtatcctacttcttgggttccatctcgtcggaaacgagatgatgttgaagcttctgagagtagtaatgaagacaggaaaaagatgcagaatgggaaaacggtggaatacccgtttgatatcagcatgataaaatccttaatccagggtcaagatgtgaaggaagcgcagaaccagaagaacaaggaagagcctggacaggttccgtatcctattttttggataccatcttatgggaaacggaaagatgttgaagcttctgagagtaaggagagtagtaatgaagggcgtaacttggagtcttgcccctctgatctccacagaaatgaagggcagataactcaagcaaaaggcaaagaagggaattttgagtgcaatgtactttcagatgcggaggagaagagctctgtaataaatatcccagtggcgaaccatctacaggaaccaagaaatattccagtgaaactatcagaaaaccatctgcctaaaccaacggagccaaccaaaaggattgcaaagaatgagccagttaagagcacaaaaaaagagcagtcttcgtcatcatcagaagcatccaagttgcctccggtttgtctgcgtgttgacccattaccgaaagagagaaatggtggttccaaatcagtaagtcatcctaaacggatggaaaaatctaaggagactaagatcgcggctccactgtcctccaagaaggctgaatcaaggactgttcctgaggcttgtaatgtaaaatgtgaagacgcaaacgcagaaatgaagatggctgagggaagcctaaacgcattaagaacagaaaaaggatcagttgagagcaattctaatcttcaagaagaatcaaacggtgagattattaaaccttgtgaagctaaggagaatagagaacagcctgcaaagaagagctttacagaagaggaagctgctagaattatccaatctatgtaccgtggatatgacgtgagaagatgggagccaattaagaaattgaaggagatagccactgtccgtgagcagatgggagatgttaaaaagcgtattgaggcgctagaagcttctactgatcagcacattgaagagaaggagattgttgttaatggagaactggtgatgaaccttcttttgaaattggatgctgtcgagggattgcatcctagtataagagagttcaggaaagctttggccacagagctctcaagcattcaagacaagcttgactccttgaaaaatagttgcgccagtgcagagaaagaggctgtaaaggaacaagtggaaattaaatctcagcctagtgactctccagtgaacttggagcactcccagctcacagaagagaacaaaatggtatctgacacgaatttagagaaggtacttcgtctgtctcccgaggaacatcctatgagcgtcttgaatagaacagatgaaaaacaggctgaatcggctgctgaaacagaagagggatatggattgtttgaaaccctggcaacggattctaagcaagctactgaaaatgcagcggctgcttcctcgacaacaattccagagaaaattggagaggttgagactgttgttccgggtaatccaccgtcagctgatggaaacggaatgacagtgaccaacgtagaggaaaacaaagccatggtggtggaaagtttggaggagccaataaacgaattgccgcaaatggtggaagagactgaaacaaactccatacgggacccggagaatgctagtgaagtctctgaagccgaaaccaactcatcagaaaatgaaaaccgaaaaggagaagatgatattgtattgcattcagaaaaaaatgtcgagctctcagagctacctgttggagtgattgacgaggaaacacagcctctttcgcaagatccatcgtcatcatatactcgtgaagggaatatgactgcaatggaccctaaaacagcgagccaggaggaaacagaagttgatcactcacccaacaactccaaaggcattggccaacagacttctgagccacaagatgaaaaagaacagtctccagagaccgaagtcattgtgaaagaacagcctctagagacagaagttattttgaacgaacaggctccggaaccggaaatcactgaacctgggatatcaaaagaaaccaaaaagctgatggaggagaaccagcgattcaaggaaacgatggagacgttggttaaggcaggcagagaacaactagaagtcatatcgaaattaaccagtcgggtcaaaagccttgagaagaaactgtcacataagaaaaagacacagatcagacgtcgtgcaagcaaaccaatgtccgtaagtccaaccgatgccgtattatga

SEQ ID NO: 4 - protein, 1043 aa, Arabidopis thaliana
MMPVYMDPSQPCQMRPQEYYYQGFGNNSQHMAMDAPPPCHGSCVHGNFPAYWPPCYPPQVPYHQCCMNRSAFHPPHASYAPSCYVHPPFPVGYQPWFDVEKDVPGKHHCGKCSSQMCDLKKDRGVVIEEHEPEIEKGEAVLPVRSTNCPYPIIWIPHENARNQEYRSSLGLGKHNQPPAEVRAPDNMTIQKSFPESWRGCFPFDESSMKSLVQNQDSKKAQNGKTVEAPFDISKFKSLLQGQDMKEAQIQKNKEELGQLTYPTSWVPSRRKRDDVEASESSNEDRKKMQNGKTVEYPFDISMIKSLIQGQDVKEAQNQKNKEEPGQVPYPIFWIPSYGKRKDVEASESKESSNEGRNLESCPSDLHRNEGQITQAKGKEGNFECNVLSDAEEKSSVINIPVANHLQEPRNIPVKLSENHLPKPTEPTKRIAKNEPVKSTKKEQSSSSSEASKLPPVCLRVDPLPKERNGGSKSVSHPKRMEKSKETKIAAPLSSKKAESRTVPEACNVKCEDANAEMKMAEGSLNALRTEKGSVESNSNLQEESNGEIIKPCEAKENREQPAKKSFTEEEAARIIQSMYRGYDVRRWEPIKKLKEIATVREQMGDVKKRIEALEASTDQHIEEKEIVVNGELVMNLLLKLDAVEGLHPSIREFRKALATELSSIQDKLDSLKNSCASAEKEAVKEQVEIKSQPSDSPVNLEHSQLTEENKMVSDTNLEKVLRLSPEEHPMSVLNRTDEKQAESAAETEEGYGLFETLATDSKQATENAAAASSTTIPEKIGEVETVVPGNPPSADGNGMTVTNVEENKAMVVESLEEPINELPQMVEETETNSIRDPENASEVSEAETNSSENENRKGEDDIVLHSEKNVELSELPVGVIDEETQPLSQDPSSSYTREGNMTAMDPKTASQEETEVDHSPNNSKGIGQQTSEPQDEKEQSPETEVIVKEQPLETEVILNEQAPEPEITEPGISKETKKLMEENQRFKETMETLVKAGREQLEVISKLTSRVKSLEKKLSHKKKTQIRRRASKPMSVSPTDAVL

SEQ ID NO: 5 - DNA, 648 bp, Arabidopis thaliana
atgaaacgttcaagaaaattctcatcgtcgacaacaacaaccaccgttattcacactttctataatgaccatactacccctccggcaaccaaagaaatcccgattgaaactccattaccggcgacgaaagccaacgtcaaaacaaacgccaccgccgcagcagcgaggattcagtctggctaccgttcttataggatccgaaacctatacaagaaaatctcatccatcaatcgggaagcgaaccgtgtacagagcataatccaacggcaagaaacagtagacgccattagaagcgatgagaaggaacgtctgagaatgaacgagactctgatggctctgcttctgaaactagactccgtccctggtttagatccgacgatcagagaagctcggaggaaagtgagccgtaagatcgtagggatgcaggaaatactcgattcaatctcggagactaaagacgaaattcaatggtggaattacaacgatctcggcggagtagactccggacaaggcggtggcgcgtggcctttgtattgggaagaagcggtggaggaagagatgtgtagagagagaggcggtgaggagatggagagattctgtgctcagtatttgggtttcagatgtttccagagatttctcagagaatga

SEQ ID NO: 6 - protein, 215 aa, Arabidopis thaliana
MKRSRKFSSSTTTTTVIHTFYNDHTTPPATKEIPIETPLPATKANVKTNATAAAARIQSGYRSYRIRNLYKKISSINREANRVQSIIQRQETVDAIRSDEKERLRMNETLMALLLKLDSVPGLDPTIREARRKVSRKIVGMQEILDSISETKDEIQWWNYNDLGGVDSGQGGGAWPLYWEEAVEEEMCRERGGEEMERFCAQYLGFRCFQRFLRE

SEQ ID NO: 7 - DNA, 2490 bp, Arabidopis thaliana
atggaggaagaggtcgtgaagtctgagaatggtagtttagagtttcatgatgacactttatcatcatcactccaggttaatggtgttcttaaagagaacgagaatccggatgttgattttcttgaggatttagattcttactgggaggacataaatgataggttaactatctcacgagtggtgagtgattcgattataaggggaatggtaactgctattgagtctgatgctgctgagaagatagctcagaaagatcttgaattgtcaaagattagggagactttgcttctgtaccatgttggttctgaagagaatgaatcctctgagtcccgtttgattcatgatgaacttactcaaggatcttcgagcagtcttaaaaagaaagcgaggaaacagttgttgatgctcgttgaagaactcaccaatttgagagagtatattcacatcaatggatcaggtgccactgtggatgattcattgggtttagacagcagtccgcatgagaccaggtccaaaactgtcgataaaatgcttgattctttgaagagcattctagagactgtgttgaagcggaagaatgatatggaacttccctcctcgtggcagcaggagcatgattttcaaaaagaaattgagtctgcagtggttactagtgttcttcggagtctcaaggatgagtatgaacagagattgttggaccaaaaagctgaatttggtggtaatagaagtctcatacttggaaatatcaaagagattactggtctgcgccaggaactagaggcgattcgtaaatcgtttttggatcatgaaaacggggacgaggcaggagaggtaggggatcggaaaagagtggagcaattacaccgcaagatgtcaggaagccttaattcagtttcttcagtttgggaaaatggtaagcatgaagagagttctactggcttaatacctgagcataacgagaccttaagacacatgtctcctgatgagatgatcaatcactttaagatcgagatgaataaaatgaaaagagaccatgattataaaatacaagagttgacagagcaatgctttacctttaagcgcaagtatctgaatttaacggaaaggggttctttttcttttgtggggaaggataaggagctaggggcgctgaaaaagaagatcccatttgtcatctctaaattggataaaattttgatggaagatgaaaagtttgtgtctgaaggcaagaatgatgctggtttaaagcgccaactggattctcttcttctggaaaatcgtcaactgaaagattcactttcagacgctgctgagaagatgtcacagctctctcaggctgaggcagatcatcaagagttgattcgaaagctcgaaacagatgttgaggattctcgtaatgaagcttctatttacgaagatgtttatgggtgttttgtgacggagtttgtgggccagattaaatgtacaaaacaggagacagatttagagcatagtatgttgagagaagcatatgaattgttattggaagatcttgcgaggaaagaagctcgtaaaagcaaggaggattttgaagactcttgtgtcaagtccgtcatgatggaagagtgttgttcagtcatatataaagaagccgtgaaggaagctcataagaaaattgttgagttgaacttgcatgtaacagaaaaagaaggtactctaagatcagagatggttgacaaggaaagactgaaagaggagattcataggctgggttgtcttgttaaggagaaggagaatttagtccaaacagctgagaataacttggctacagagagaaagaaaattgaggtagtctctcaacagattaacgatctgcaatctcaggtggaacggcaagaaacagaaattcaggataagatagaagcactgagcgttgtttcggcacgcgagttagagaaagtaaaaggttatgagacgaagatatccagcttgagagaagagttggaactagcaagagagagtttgaaggaaatgaaagatgaaaaaaggaaaaccgaggaaaagttatcagagacaaaagcagagaaagagacacttaagaagcaacttgtgtctctggacttagtcgttcctcctcaattgataaaagggttcgacattctagagggtttgatagcagaaaagacgcaaaaaacgaattctaggttgaaaaacatgcagagtcaactgagtgatctgtcacatcagatcaatgaagtcaaggggaaagcatctacgtacaagcaacggctggagaagaagtgttgtgacctcaagaaggctgaggctgaggttgatcttcttggagatgaggttgaaactcttttggatcttcttgagaaaatatatatcgctctcgatcattactctccaatcctaaagcattaccctggcgtaagcacgatctcggtctacacatatttcatttttctttccattttgagattgattttaatgatgagaccatactga

SEQ ID NO: 8 - protein, 829 aa, Arabidopis thaliana
MEEEVVKSENGSLEFHDDTLSSSLQVNGVLKENENPDVDFLEDLDSYWEDINDRLTISRVVSDSIIRGMVTAIESDAAEKIAQKDLELSKIRETLLLYHVGSEENESSESRLIHDELTQGSSSSLKKKARKQLLMLVEELTNLREYIHINGSGATVDDSLGLDSSPHETRSKTVDKMLDSLKSILETVLKRKNDMELPSSWQQEHDFQKEIESAVVTSVLRSLKDEYEQRLLDQKAEFGGNRSLILGNIKEITGLRQELEAIRKSFLDHENGDEAGEVGDRKRVEQLHRKMSGSLNSVSSVWENGKHEESSTGLIPEHNETLRHMSPDEMINHFKIEMNKMKRDHDYKIQELTEQCFTFKRKYLNLTERGSFSFVGKDKELGALKKKIPFVISKLDKILMEDEKFVSEGKNDAGLKRQLDSLLLENRQLKDSLSDAAEKMSQLSQAEADHQELIRKLETDVEDSRNEASIYEDVYGCFVTEFVGQIKCTKQETDLEHSMLREAYELLLEDLARKEARKSKEDFEDSCVKSVMMEECCSVIYKEAVKEAHKKIVELNLHVTEKEGTLRSEMVDKERLKEEIHRLGCLVKEKENLVQTAENNLATERKKIEVVSQQINDLQSQVERQETEIQDKIEALSVVSARELEKVKGYETKISSLREELELARESLKEMKDEKRKTEEKLSETKAEKETLKKQLVSLDLVVPPQLIKGFDILEGLIAEKTQKTNSRLKNMQSQLSDLSHQINEVKGKASTYKQRLEKKCCDLKKAEAEVDLLGDEVETLLDLLEKIYIALDHYSPILKHYPGVSTISVYTYFIFLSILRLILMMRPY

SEQ ID NO: 9 - DNA, 19 bp, Artificial Sequence
  PCR primer
gggaggtgagacctggtgg

SEQ ID NO: 10 - DNA, 21 bp, Artificial Sequence
  PCR primer
ggccgaaggcgaagctggcgg